(19) United States
(12) Patent Application Publication
(10) Pub. No.: US 2026/0098241 A1
(43) Pub. Date: Apr. 9, 2026

(54) RECOMBINANT YEAST, METHOD FOR PRODUCING TARGET PROTEIN, KIT, AND METHOD FOR PRODUCING RECOMBINANT YEAST

(71) Applicant: FUJIFILM Corporation, Tokyo (JP)

(72) Inventors: Arjo Lysander DE BOER, Tilburg (NL); Hidekazu SAKAI, Ashigarakami-gun (JP); Sayami FUJIWARA, Ashigarakami-gun (JP); Kazuto FUKUNAGA, Ashigarakami-gun (JP); Takahiro HIRATSUKA, Ashigarakami-gun (JP)

(73) Assignee: FUJIFILM Corporation, Tokyo (JP)

(21) Appl. No.: 19/410,452

(22) Filed: Dec. 5, 2025

Related U.S. Application Data
(63) Continuation of application No. PCT/JP2024/020485, filed on Jun. 5, 2024.

(30) Foreign Application Priority Data
Jun. 6, 2023 (JP) 2023-092875

(51) Int. Cl. C12N 1/16; C12N 9/24

(52) U.S. Cl. CPC C12N 1/16; C12N 9/2402; C12Y 302/0102

(57) ABSTRACT

An object of the present invention is to provide a recombinant yeast that can highly express a target protein in a tightly regulated manner without using methanol, and to provide a method for producing a target protein using the recombinant yeast, a kit for producing the recombinant yeast, and a method for producing the recombinant yeast. According to the present invention, there is provided a recombinant yeast containing a first exogenous sucrose-inducible promoter for expressing a target protein.

### CROSS-REFERENCE TO RELATED APPLICATIONS

This application is a Continuation of PCT International Application No. PCT/JP2024/020485 filed on Jun. 5, 2024, which claims priority under 35 U.S.C § 119 (a) to Japanese Patent Application No. 2023-092875 filed on Jun. 6, 2023. Each of the above application(s) is hereby expressly incorporated by reference, in its entirety, into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 5, 2025, is named “2870-0896PUS1.xml” and is 63,946 bytes in size. The sequence listing contained in this .XML file is part of the specification and is hereby incorporated by reference herein in its entirety.

### BACKGROUND OF THE INVENTION

### 1. Field of the Invention

The present invention relates to a recombinant yeast for expressing a target protein. The present invention further relates to a method for producing a target protein using the recombinant yeast, a kit for producing the recombinant yeast, and a method for producing the recombinant yeast.

### 2. Description of the Related Art

Recombinant proteins are useful as a pharmaceutical or an industrial enzyme, and are produced by a recombinant microorganism in many cases. Methanol-assimilating yeasts have been utilized as an efficient protein expression system. As methanol-assimilating yeasts that enables high expression of a protein and high-density culture, Ogataea polymorpha, Candida boidinii, Ogataea methanolica, and Pichia pastoris (Komagataella phaffii) are known. In particular, Pichia pastoris has been extensively utilized in commercial applications.

As a promoter for expressing a protein in Pichia pastoris, an AOX1 promoter has been utilized. The AOX1 promoter is a tightly regulated methanol-inducible promoter, and enables strong inducible expression using methanol as a carbon source.

Furthermore, in recent years, as an alternative promoter that does not use methanol, an AOX1 promoter mutant (WO2006/089329A), a G1 promoter mutant (WO2017/021541A), an FMD/MOX promoter (WO2017/109082A), and the like have been developed.

### SUMMARY OF THE INVENTION

In the use of the AOX1 promoter, methanol is used. However, in a case of using methanol which is flammable and toxic, it is necessary to take explosion-proof measures and to address health risks. Therefore, there is a problem that expensive equipment is required in particular in a case of industrial use on a large scale. In addition, by using methanol as a carbon source, the survival rate of the cell is reduced as compared with a carbon source such as glucose, which may affect the yield of the product. Therefore, even in a case where an increase in protein expression is desired, the amount of the carbon source to be added is limited. Furthermore, heat generation due to oxygen consumption and metabolism is large, and the increase in cooling cost is also a problem.

On the other hand, the promoters described in WO2006/089329A, WO2017/021541A, and WO2017/109082A have a problem that the expression ability is lower than or equal to that of the AOX1 promoter, or it is difficult to achieve tight regulation of expression because of the expression under repression-release conditions. In addition, in a case of expressing various exogenous genes to improve the productivity of the protein (for example, in a case where protein A is expressed to function as a chaperone for target protein B), a plurality of types of alternative promoters are required, but there is a limitation in the number in a case of using existing promoters.

Therefore, a novel promoter that does not use methanol, tightly regulates expression, and has a high expression level is required.

An object to be achieved by the present invention is to provide a recombinant yeast that can highly express a target protein in a tightly regulated manner without using methanol. Another object to be achieved by the present invention is to provide a method for producing a target protein using the recombinant yeast, a kit for producing the recombinant yeast, and a method for producing the recombinant yeast.

As a result of intensive studies to achieve the above object, the present inventors have found that, by using an exogenous sucrose-inducible promoter as a promoter for expressing a target protein in a yeast, the target protein can be highly expressed in a tightly regulated manner without using methanol. The present invention has been completed based on the above findings.

According to an aspect of the present invention, the following invention is provided.

<1> A recombinant yeast comprising a first exogenous sucrose-inducible promoter for expressing a target protein.

<2> The recombinant yeast according to <1>, further comprising an exogenous gene encoding the target protein.

<3> The recombinant yeast according to <1> or <2>, further comprising an exogenous gene that confers sucrose assimilation.

<4> The recombinant yeast according to <3>, in which the exogenous gene that confers sucrose assimilation is linked to a second exogenous sucrose-inducible promoter.

<5> The recombinant yeast according to <3> or <4>, in which the exogenous gene that confers sucrose assimilation is a maltase gene.

<6> The recombinant yeast according to any one of <3> to <5>, in which the first exogenous sucrose-inducible promoter and the exogenous gene that confers sucrose assimilation are derived from a yeast having an α-glucosidase which has sucrose-hydrolyzing activity.

<7> The recombinant yeast according to <6>, in which the yeast having an α-glucosidase which has sucrose-hydrolyzing activity belongs to any of the genus Ogataea, Candida, Cyberlindnera, Wickerhamomyces, Clavispora, Debaryomyces, Meyerozyma, Scheffersomyces, Metschnikowia, Spathaspora, Babjeviella, Hypopichia, or Yamadazyma.

<8> The recombinant yeast according to <7>, in which the yeast having an α-glucosidase which has sucrose-hydrolyzing activity is Ogataea parapolymorpha.

<9> The recombinant yeast according to any one of <1> to <8>, in which the recombinant yeast is the genus Komagataella.

<10> A method for producing a target protein, comprising culturing the recombinant yeast according to any one of <1> to <9> in a culture medium containing sucrose as a carbon source.

<11> The method for producing a target protein according to <10>, in which the target protein is an antibody, a bioactive protein, a bioactive polypeptide, an extracellular matrix, an artificial protein, or an enzyme.

<12> The method for producing a target protein according to <10> or <11>, in which a concentration of the sucrose in the culture medium during the culture is 0.5 to 2 g/mL.

<13> The method for producing a target protein according to any one of <10> to <12>, in which an addition rate of the sucrose is 2 to 100 g/hour per 0.9 L of the culture medium at the start of the culture.

<14> A kit for producing the recombinant yeast according to any one of <1> to <9>, the kit comprising a yeast and an expression construct containing a sucrose-inducible promoter.

<15> The kit according to <14>, in which the expression construct contains an exogenous gene encoding the target protein, and the sucrose-inducible promoter induces expression of the exogenous gene encoding the target protein.

<16> A method for producing a recombinant yeast, which is a method for producing the recombinant yeast according to any one of <1> to <9>, the method comprising introducing an expression construct containing a sucrose-inducible promoter into a yeast.

<17> The method for producing a recombinant yeast according to <16>, in which the expression construct containing the sucrose-inducible promoter is introduced into a genome of the yeast.

<18> The method for producing a recombinant yeast according to <16> or <17>, in which the expression construct containing the sucrose-inducible promoter contains an exogenous gene encoding the sucrose-inducible promoter and the target protein, and the sucrose-inducible promoter is an expression construct that induces expression of an exogenous gene encoding the target protein.

<19> The method for producing a recombinant yeast according to <16>, in which the expression construct containing the sucrose-inducible promoter is introduced into a genome of a yeast having an exogenous gene that confers sucrose assimilation.

According to the present invention, the target protein can be highly expressed in a tightly regulated manner without using methanol.

### BRIEF DESCRIPTION OF THE DRAWINGS

FIG. 1 shows a MAL cluster region for converting an AOX2 CDS of Pichia pastoris. The cluster region includes AG1, AGT1, and MAL-activator, and has sucrose assimilation.

FIG. 2 shows a relationship between product A and product B.

FIG. 3 shows screening of a MAL cluster-introduced yeast strain using a YNB-sucrose-supplemented medium. Left: Colony formation of the MAL cluster-introduced yeast strain was observed. Right: No colony was formed in the MAL cluster non-introduced yeast strain.

FIG. 4 shows a construction of a plasmid vector having the produced sucrose-inducible promoter.

FIG. 5 is a schematic diagram showing that a linearized plasmid is inserted into a MAL1 site on the MAL cluster by homologous recombination.

FIG. 6 shows a construction of a plasmid vector having the produced methanol-inducible promoter.

### DESCRIPTION OF THE PREFERRED EMBODIMENTS

Hereinafter, an example of the embodiment of the present disclosure will be described. However, the present disclosure is not limited to the following embodiments, and can be implemented with appropriate changes within the scope of the object of the present disclosure. In the present specification, a numerical range indicated using “to” represents a range including numerical values described before and after “to” as a minimum value and a maximum value.

### <Recombinant Yeast>

The recombinant yeast according to the embodiment of the present invention contains a first exogenous sucrose-inducible promoter for expressing a target protein.

The first exogenous sucrose-inducible promoter in the present invention is a promoter for expressing a target protein. The first exogenous sucrose-inducible promoter is located upstream of a gene encoding a target protein to induce expression of the gene encoding the target protein. By using the sucrose-inducible promoter, the target protein can be expressed in a tightly regulated manner.

The gene encoding the target protein may be a gene originally present in the yeast or an exogenous gene, but is preferably the exogenous gene. That is, the recombinant yeast according to the embodiment of the present invention preferably further contains an exogenous gene encoding a target protein.

The first exogenous sucrose-inducible promoter is not particularly limited, and for example, a promoter of an α-glucosidase gene, an α-glucoside permease gene, or a MAL-activator gene contained in a MAL cluster described in Katrin Viigand et al., Genome Mining of Non-Conventional Yeasts: Search and Analysis of MAL Clusters and Proteins, Genes 2018, 9, 354; doi: 10.3390/genes9070354 can be used. Details of the α-glucosidase gene, the α-glucoside permease gene, or the MAL-activator gene will be described later.

The first exogenous sucrose-inducible promoter is more preferably a promoter of an α-glucosidase gene and still more preferably a promoter of an AG1 gene among the above. Specific examples thereof include a promoter having a nucleotide sequence set forth in SEQ ID NO: 16.

The promoter of the AG1 gene may be a promoter having a nucleotide sequence having a sequence identity of 80% or more, preferably 85% or more, more preferably 90% or more, still more preferably 95% or more, and particularly preferably 98% or more with the nucleotide sequence set forth in SEQ ID NO: 16 as long as it has promoter activity.

The sequence identity refers to a value calculated by the following equation.

% Sequence identity=[(number of identical bases)/(alignment length)]×100

The sequence identity between two nucleotide sequences can be determined by any method known to those skilled in the art, and can be determined using the Basic Local Alignment Search Tool (BLAST) program (J. Mol. Biol. 215:403-410, 1990) or the like.

The recombinant yeast according to the embodiment of the present invention preferably further includes an exogenous gene that confers sucrose assimilation.

As the exogenous gene that confers sucrose assimilation, a gene encoding an enzyme involved in sucrose assimilation, a transporter gene involved in sucrose conversion, a gene encoding a transcriptional activator of the gene encoding an enzyme involved in the sucrose assimilation, or the like can be used.

Specific examples of the exogenous gene that confers sucrose assimilation include an α-glucosidase gene, an α-glucoside permease gene, a MAL-activator gene (such as those described in Genes 2018, 9, 354; doi: 10.3390/genes9070354), and the like.

Examples of the α-glucosidase gene include a maltase gene, an isomaltase gene, a sucrase gene and the like. Specific examples of the maltase gene (including a gene having a function of the isomaltase gene in part) include MAL32 (S. cerevisiae), MAL1 (O. polymorpha), AG1 (O. parapolymorpha), AG1 (L. elongisporus), AG2 (M. guiliermondii), AG1.2 (C. fabianii), AGL1 (S. stipitis), MAL6 (S. stipitis), MAL7 (S. stipitis), MAL8 (S. stipitis), MalT (A. oryzae), AG1 (L. starkeyi), AG2 (L. starkeyi), AG4 (L. starkeyi), AG5 (L. starkeyi), AG6 (L. starkeyi), and examples of the isomaltase gene include AG1 (T. delbrueckii), AG1 (M. guiliermondii), AG1.1 (C. fabianii), AG3 (L. starkeyi), AG7 (L. starkeyi), AG8 (L. starkeyi), and the like.

Examples of the α-glucoside permease gene include MAL1 (S. stipitis), MAL2 (O. polymorpha), MAL2 (S. stipitis), MAL3 (S. stipitis), MAL4 (S. stipitis), MAL5 (S. stipitis), MAL31 (S. cerevisiae), MalP (A. oryzae), AGT1 (O. parapolymorpha), AGT1.1 (M. guiliermondii), AGT1.2 (M. guiliermondii), AGT1.3 (M. guiliermondii), AGT2.1 (L. starkeyi), AGT2.2 (L. starkeyi), AGT3 (L. starkeyi), AGT4 (L. starkeyi), AGT5 (L. starkeyi), AGT6 (L. starkeyi), and the like.

Examples of the MAL-activator gene include MAL33 (S. cerevisiae), MAL-ACT (O. parapolymorpha), MAL-ACT1 (O. polymorpha), MAL-ACT2 (O. polymorpha), MalR (A. oryzae), SUC1.1 (S. stipitis), SUC1.2 (S. stipitis), SUC1.4 (S. stipitis), and the like.

It is preferable that the exogenous gene that confers sucrose assimilation is linked to the second exogenous sucrose-inducible promoter.

The second exogenous sucrose-inducible promoter may be the same promoter as the first exogenous sucrose-inducible promoter or may be a different promoter from the first exogenous sucrose-inducible promoter, but it is preferably the same promoter as the first exogenous sucrose-inducible promoter.

Specific examples of the second exogenous sucrose-inducible promoter include the same examples as those described for the first exogenous sucrose-inducible promoter.

The first sucrose-inducible promoter and the exogenous gene that confers sucrose assimilation are preferably derived from a yeast having an α-glucosidase which has sucrose-hydrolyzing activity.

Examples of the yeast having an α-glucosidase which has sucrose-hydrolyzing activity include a yeast belonging to any of the genus Ogataea, Candida, Cyberlindnera, Wickerhamomyces, Clavispora, Debaryomyces, Meyerozyma, Scheffersomyces, Metschnikowia, Spathaspora, Babjeviella, Hypopichia, or Yamadazyma, but the yeast is not particularly limited. The yeast having an α-glucosidase which has sucrose-hydrolyzing activity is more preferably the genus Ogataea and particularly preferably Ogataea parapolymorpha.

In a case where the recombinant yeast according to the embodiment of the present invention has the exogenous gene that confers sucrose assimilation, a positional relationship between the gene encoding the target protein and the exogenous gene that confers sucrose assimilation is not particularly limited. The gene encoding the target protein may be inserted into the genome of the recombinant yeast or may be present outside the chromosome. The exogenous gene that confers sucrose assimilation may also be inserted into the genome of the recombinant yeast or may be present outside the chromosome. In a case where the gene encoding the target protein and the exogenous gene that confers sucrose assimilation are inserted into the genome of the recombinant yeast, insertion positions of the genes on the genome are not particularly limited. The gene encoding the target protein and the exogenous gene that confers sucrose assimilation may be inserted at distant positions on the genome or may be present at adjacent positions to each other. In a case where the gene encoding the target protein and the exogenous gene that confers sucrose assimilation are present at adjacent positions to each other, the gene encoding the target protein may be present upstream or downstream of the exogenous gene that confers sucrose assimilation.

Examples of the recombinant yeast according to the embodiment of the present invention include a yeast belonging to Komagataella, Pichia, Ogataea, Candida, Saccharomyces, Torulopsis, Zygosaccharomyces, Schizosaccharomyces, Yarrowia, Kluyveromyces, Debaryomyces, Geotrichum, Wickerhamomyces, Fellomyces, Sporobolomyces, and the like.

In the genus Komagataella, Komagataella pastoris, Komagataella phaffii, or the like is preferable. Both Komagataella pastoris and Komagataella phaffii have the synonym Pichia pastoris.

In the genus Pichia, Pichia methanolica or the like is preferable. In the genus Ogataea, Ogataea angusta, Ogataea polymorpha, Ogataea parapolymorpha, Ogataea minuta, or the like is preferable. In the genus Candida, Candida boidinii or the like is preferable.

Among the above, the recombinant yeast according to the embodiment of the present invention is particularly preferably the genus Komagataella. Specific examples of the strain that can be used include Komagataella pastoris ATCC 76273 (Y-11430, Pichia pastoris CBS7435). This strain can be obtained from American Type Culture Collection, Thermo Fisher Scientific, Inc., or the like.

### <Kit for Producing Recombinant Yeast>

The kit for producing the recombinant yeast according to the embodiment of the present invention includes a yeast and an expression construct containing a sucrose-inducible promoter.

As the yeast, the yeast belonging to the above-described genus for the recombinant yeast can be used, but the yeast is not particularly limited. The yeast is particularly preferably the genus Komagataella.

The expression construct preferably contains an exogenous gene encoding a target protein, and the above-described sucrose-inducible promoter induces expression of the exogenous gene encoding the target protein.

The expression construct containing the sucrose-inducible promoter can be obtained by inserting the sucrose-inducible promoter into an appropriate vector. The vector for inserting the sucrose-inducible promoter is not particularly limited as long as it is replicable in the host, and examples thereof include a plasmid, a phage DNA, a virus vector, and the like. In addition, in a case where the vector itself does not have a replication ability, a DNA fragment that can be replicated by being inserted into a chromosome of the host may be used.

Examples of the plasmid include a plasmid derived from Escherichia coli (for example, pBR322, pBR325, pUC118, pUC119, pUC18, pUC19, pBluescript, and the like), a plasmid derived from Bacillus subtilis (for example, pUB110, pTP5, and the like), a plasmid derived from yeast (for example, a YEp series such as YEp13, a YRp series, a Yip series, a YCp system such as YCp50, and the like, a Pichia vector system such as pPIC9, pPICZ, and pPICZα), and the like. Examples of the phage DNA include λ phage (Charon 4A, Charon 21A, EMBL3, EMBL4, λgt10, λgt11, λZAP, and the like). Furthermore, an animal virus such as a retrovirus or a vaccinia virus, or an insect virus vector such as a baculovirus can also be used.

To insert the sucrose-inducible promoter into the vector, first, the purified DNA may be cut with an appropriate restriction enzyme and then inserted into a restriction enzyme site or a multi-cloning site of an appropriate vector DNA to be linked to the vector. Alternatively, the vector and the sucrose-inducible promoter may be linked to each other by an in vitro method using PCR or the like or an in vivo method using yeast or the like by providing a region having homology to each other in a part of the vector and a part of the sucrose-inducible promoter.

It is preferable that the expression construct containing the sucrose-inducible promoter further contains an exogenous gene encoding a target protein downstream of the sucrose-inducible promoter. The method of inserting the exogenous gene is the same as the method of inserting the sucrose-inducible promoter into the vector.

### <Method for Producing Recombinant Yeast>

The method for producing the recombinant yeast according to the embodiment of the present invention includes introducing an expression construct containing a sucrose-inducible promoter into a yeast.

As the expression construct containing the sucrose-inducible promoter, and the yeast, those described above in the present specification can be used. As the expression construct containing the sucrose-inducible promoter, it is preferable that the expression construct contains the sucrose-inducible promoter and an exogenous gene encoding a target protein, and the sucrose-inducible promoter induces expression of the exogenous gene encoding the target protein.

The method of introducing the expression construct containing the sucrose-inducible promoter into the yeast is not particularly limited as long as it is a method of introducing DNA into the yeast, and examples thereof include electroporation, spheroplast method, lithium acetate method, and the like.

The expression construct containing the sucrose-inducible promoter may be introduced into the genome of the yeast, or the expression construct containing the sucrose-inducible promoter may be introduced into the yeast as extrachromosomal DNA, but is preferably introduced into the genome of the yeast. The introduction into the genome of the yeast may be performed by homologous recombination or may be performed in an aspect other than homologous recombination.

In a preferred aspect of the present invention, the expression construct containing the sucrose-inducible promoter is introduced into the yeast having the exogenous gene that confers sucrose assimilation. Preferably, the expression construct containing the sucrose-inducible promoter is introduced into the genome of the yeast having the exogenous gene that confers sucrose assimilation.

The presence or absence of the incorporation of the sucrose-inducible promoter into the yeast can be confirmed by a polymerase chain reaction (PCR) method, a Southern hybridization method, or the like. For example, DNA is prepared from the transformant, a DNA-specific primer is designed, and PCR is performed. Thereafter, the amplification product is subjected to agarose gel electrophoresis, polyacrylamide gel electrophoresis, capillary electrophoresis, or the like, is stained with ethidium bromide or the like, and is detected as one band, whereby it can be confirmed that the transformation has been performed.

In the recombinant yeast according to the embodiment of the present invention, selection can be performed by introducing an antibiotic resistance gene as a selection marker in advance, culturing the yeast using a culture medium containing the antibiotic, and acquiring a colony that has proliferated. As the antibiotic, zeocin, kanamycin, hygromycin, puromycin, blasticidin, or the like can be used.

The recombinant yeast into which the expression construct containing the sucrose-inducible promoter is introduced, which is produced as described above, can be cultured and grown by an appropriate conventional method and then used in the method for producing a target protein.

### <Method for Producing Target Protein>

The method for producing a target protein according to the embodiment of the present invention includes culturing the recombinant yeast according to the embodiment of the present invention in a culture medium containing sucrose as a carbon source.

As the sucrose source, biomass, a chemically synthesized product, or the like can be used, but the sucrose source is not particularly limited.

A concentration of the sucrose in the culture medium during the culture is preferably 0.5 to 2 g/mL, more preferably 0.5 to 0.7 g/mL, and still more preferably 0.55 to 0.65 g/mL.

A addition rate of the sucrose is preferably 2 to 100 g/hour, more preferably 2 to 20 g/hour, and still more preferably 3 to 15 g/hour per 0.9 L of the culture medium at the start of the culture.

Examples of the target protein include an antibody, a bioactive protein, a bioactive polypeptide, an extracellular matrix, an artificial protein, an enzyme, and the like, but the target protein is not particularly limited.

The antibody is a heterotetrameric protein in which two polypeptide chains of L chains and two polypeptide chains of H chains are linked by a disulfide bond, and is not particularly limited as long as it has an ability to bind to a specific antigen. The antibody may be a partial antibody (fragmented antibody). Examples of the partial antibody include an Fab antibody, an (Fab)2 antibody, an scFv antibody, a diabody, and the like, but the partial antibody is not particularly limited. The antibody is preferably a human antibody, a humanized antibody, a chimeric antibody, a mouse antibody, a bispecific antibody, or the like. The class of the antibody is also not particularly limited, and may be any one class of IgG such as IgG1, IgG2, IgG3, and IgG4, IgA, IgD, IgE, or IgM, but in a case of being used as a medicine, IgG and IgM are preferable.

Examples of the bioactive protein and the bioactive polypeptide include a hormone, a growth factor, a coagulation factor, a neuroprotein, an apolipoprotein, a tumor suppressor, an antigen including an antigenic protein and peptide of an endogenous or exogenous tumor, a bacterial surface protein and peptide, a viral surface protein and peptide, a protozoan cell surface protein and peptide, a fungal surface protein and peptide, and a parasitic substance including a viral reverse transcriptase and a related viral specific enzyme. Examples of the other proteins include a receptor such as an insulin receptor, a hormone receptor, and a growth factor receptor. In Examples described later, recombinant gelatin was used as the target protein.

Examples of the enzyme include an enzyme derived from a microorganism and an enzyme produced by an animal or a plant. Examples of the enzyme include phytase, amylase, glucosidase, cellulase, lipase, protease, glutaminase, peptidase, nuclease, oxidase, lactase, xylanase, trypsin, pectinase, isomerase, and the like, but the enzyme is not limited thereto.

The culture of the recombinant yeast can be performed using a normal method of culturing yeast, and the culture conditions can be appropriately set under conditions suitable for the growth of the yeast.

The culture medium used for the culture may be any of a natural medium or a synthetic medium as long as it contains a carbon source including sucrose, a nitrogen source, an inorganic substance, and a trace nutrient required by the used strain as necessary.

The carbon source of the culture medium may include sucrose and may include a carbon source other than sucrose. As the carbon source other than sucrose, for example, a sugar such as glucose, maltose, fructose, mannose, trehalose, mannitol, sorbitol, starch, dextrin, or molasses, an organic acid such as citric acid or succinic acid, glycerol, or the like can be used.

As the nitrogen source of the culture medium, for example, an organic nitrogen source such as corn steep liquor, soybean meal, or peptone, or an inorganic nitrogen source such as ammonium chloride, ammonium sulfate, urea, ammonium nitrate, sodium nitrate, ammonium phosphate, or ammonia can be used. Furthermore, a nitrogen-containing natural substance such as peptone, polypeptone, bacto peptone, meat extract, fish extract, yeast extract, corn steep liquor, soybean flour, soybean meal, dried yeast, casamino acids, or soluble vegetable protein can also be used as the nitrogen source.

As the inorganic substance of the culture medium, for example, a calcium salt, a magnesium salt, a potassium salt, a sodium salt, a phosphate salt, a manganese salt, a zinc salt, an iron salt, a copper salt, a molybdenum salt, a cobalt salt, or the like is appropriately used. Specifically, potassium dihydrogen phosphate, dipotassium hydrogen phosphate, magnesium sulfate, ferrous sulfate, manganese sulfate, zinc sulfate, sodium chloride, potassium chloride, calcium chloride, or the like is used. Furthermore, phosphoric acid, sulfuric acid, potassium hydroxide, Trace Element Solution (TES), or the like is appropriately used.

As the culturing method, a liquid culturing method is preferable, and any of batch culture, fed-batch culture, continuous culture, or perfusion culture may be used.

The culture temperature and pH may be conditions suitable for the proliferation of the recombinant yeast. The temperature is preferably 20° C. to 40° C. and more preferably 25° C. to 35° C. The pH is preferably 2 to 9 and more preferably 5 to 8.

The culture time is preferably 1 hour to 14 days, more preferably 6 hours to 10 days, and still more preferably 12 hours to 7 days.

The culture can be preferably performed by shaking or aeration stirring.

In a case of secreting a non-secretory target protein extracellularly from yeast, a nucleotide sequence encoding a signal sequence can be introduced to the 5′ end of the target protein gene. The nucleotide sequence encoding the signal sequence is not particularly limited as long as it is a nucleotide sequence encoding a signal sequence that can be secreted and expressed by the yeast. Specific examples of the nucleotide sequence encoding the signal sequence include a nucleotide sequence encoding a signal sequence of mating factor α (MFα) of Saccharomyces cerevisiae, acid phosphatase (PHO1) of Ogataea angusta, acid phosphatase (PHO1) of Komagataella pastoris, invertase (SUC2) of Saccharomyces cerevisiae, PLB1 of Saccharomyces cerevisiae, bovine serum albumin (BSA), human serum albumin (HSA), and an immunoglobulin.

By culturing the recombinant yeast according to the embodiment of the present invention, the target protein can be accumulated in the host or the culture solution and recovered. As a method of recovering the target protein, known purification methods can be appropriately combined and used. For example, the recombinant yeast is cultured in a culture medium, and the cells are removed from the culture supernatant by centrifugation or filtration of the culture solution. The target protein can be recovered from the obtained culture supernatant by using a method such salting out (ammonium sulfate precipitation, sodium phosphate precipitation, or the like), solvent precipitation (protein fractionation precipitation method using acetone or ethanol or the like), dialysis, gel filtration chromatography, ion exchange chromatography, hydrophobic chromatography, affinity chromatography, reversed-phase chromatography, or ultrafiltration alone or in combination.

The present invention will be more specifically described using the following examples; however, it is not limited by the examples.

### EXAMPLES

### <Example 1> Cloning of MAL Cluster

The genome of Ogataea Parapolymorpha containing the MAL cluster having SEQ ID NO: 1 and the genome of Pichia pastoris CBS7435 having AOX2 were used as a template.

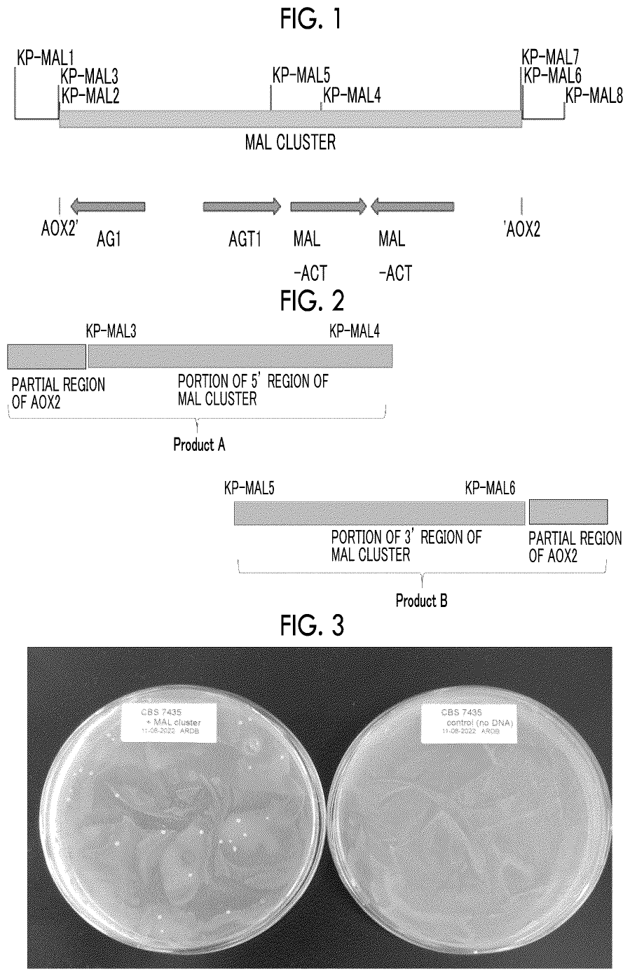

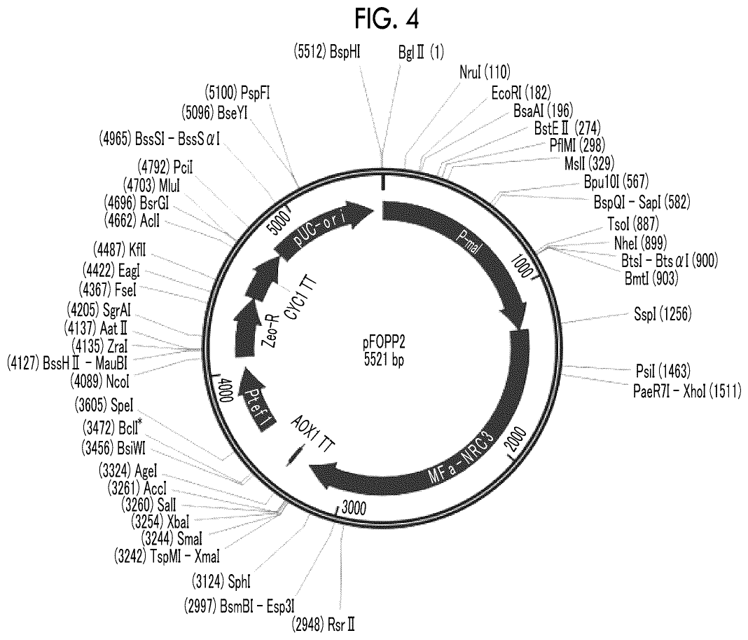

As shown in FIG. 1, using KP-MAL3 and KP-MAL4, the 5′ region of the MAL cluster was amplified using the genome of Ogataea parapolymorpha as a template. The nucleotide sequence of the amplified fragment is set forth in SEQ ID NO: 2. Using KP-MAL1 and KP-MAL2, the partial region of AOX2 was amplified using the genome of Pichia pastoris CBS7435 having AOX2 as a template. The nucleotide sequence of the amplified fragment is set forth in SEQ ID NO: 3. In addition, using the two amplification products obtained above, a concatenated amplification product was obtained by a PCR reaction (product A).

Similarly, using KP-MAL5 and KP-MAL6, the 3′ region of the MAL cluster was amplified using the genome of Ogataea Parapolymorpha as a template. The nucleotide sequence of the amplified fragment is set forth in SEQ ID NO: 4. Using KP-MAL7 and KP-MAL8, the partial region of AOX2 was amplified using the genome of Pichia pastoris CBS7435 having AOX2 as a template. The nucleotide sequence of the amplified fragment is set forth in SEQ ID NO: 5. Using the two amplification products obtained above, a concatenated amplification product was obtained by a PCR reaction (product B).

The relationship between product A and product B is shown in FIG. 2.

### Primers for MAL Cluster Amplification

KP-MAL1 (Forward):
(SEQ ID NO: 6)
5′-TTGTCAGCTTAAAGGACTCC-3′
KP-MAL2 (Reverse):
(SEQ ID NO: 7)
5′-TTCCGGGAATACCATTTTTCTCAGTTGATTTGTTTGTGGG-3′
KP-MAL3 (Forward):
(SEQ ID NO: 8)
5′-CAAATCAACTGAGAAAAATGGTATTCCCGGAAAGAACTCG-3′
KP-MAL4 (Reverse):
(SEQ ID NO: 9)
5′-CCTGGTCTTCTGTGAGTATC-3′
KP-MAL5 (Forward):
(SEQ ID NO: 10)
5′-TGGGCAGGCTTGTCTGTTTG-3′
KP-MAL6 (Reverse):
(SEQ ID NO: 11)
5′-CATAGATACAACATAAACTAGGAATGTTTCCAGCGGTATG-3′
KP-MAL7 (Forward):
(SEQ ID NO: 12)
5′-CATACCGCTGGAAACATTCCTAGTTTATGTTGTATCTATG-3′
KP-MAL8 (Reverse):
(SEQ ID NO: 13)
5′-CTGACTGGACTTAGCGAAG-3′

The product A and the product B (each sample amount of 2 to 10 μg) obtained above and Pichia pastoris CBS7435 (OD600: 200 to 300, 60 μL) were simultaneously introduced into Pichia pastoris CBS7435 by electroporation (MicroPulser electroporator (Bio-Rad)) to perform the transformation of Pichia pastoris CBS7435. As a result, the product A and the product B were inserted into the AOX2 site of the genome of Pichia pastoris by homologous recombination. To confirm that the MAL cluster composed of the product A and the product B was introduced into the CDS of the AOX2 site by homologous recombination, the screening was performed using a YNB-sucrose-supplemented medium, and a colony in which the MAL cluster was incorporated was acquired (FIG. 3).

### <Example 2> Production of Plasmid Vector in which Gene of Target Protein is Linked Downstream of Sucrose-Inducible MAL Promoter

As a model protein, recombinant gelatin CBE3 described below was used. (Described in WO2008/103041A).
Molecular weight: 51.6 kD
Structure: GAP[(GXY)₆₃]3G
Number of amino acids: 571 amino acids
RGD sequence: 12 sequences
Imino acid content: 33%
Almost 100% of amino acids have a repeating structure of GXY.
CBE3 has an ERGD sequence.

The amino acid sequence of the CBE3 does not include a serine residue, a threonine residue, an asparagine residue, a tyrosine residue, and a cysteine residue.
Isoelectric point: 9.34
Hydrophilic repeating unit ratio in polymer: 26.1%

The amino acid sequence is set forth in SEQ ID NO: 14.

(SEQ ID NO: 14)
GAP(GAPGLQGAPGLQGMPGERGAAGLPGPKGERGDAGPKGA
DGAPGAPGLQGMPGERGAAGLPGPKGERGDAGPKGADGAPGK
DGVRGLAGPIGPPGERGAAGLPGPKGERGDAGPKGADGAPGK
DGVRGLAGPIGPPGPAGAPGAPGLQGMPGERGAAGLPGPKGE
RGDAGPKGADGAPGKDGVRGLAGPP)3G

A plasmid vector in which a gene encoding a target protein and a zeocin resistance gene were linked to the downstream of the sucrose-inducible MAL promoter was produced.

The nucleotide sequence of P-mal is set forth in SEQ ID NO: 16.
MF-a: signal sequence of target protein is shown in SEQ ID NO: 17.
NRC3: nucleotide sequence encoding target protein (CBE3) is set forth in SEQ ID NO: 18.

### <Example 3> Transformation of Pichia pastoris

The plasmid vector (plasmid vector having a sucrose-inducible promoter) produced in Example 2 was introduced into the Pichia pastoris CBS7435 strain produced in Example 1, by homologous recombination. Specifically, the plasmid vector (produced in Example 2) of SEQ ID NO: 15 in which a gene of a target protein was linked to the downstream of the sucrose-inducible MAL promoter was linearized with a restriction enzyme EcoRI. The linearized plasmid (amount of linearized plasmid of 2 to 10 μg) was introduced into the Pichia pastoris CBS7435 strain (OD600: 200 to 300, 60 μL) produced in Example 1 by electroporation. The electroporation was performed using an electroporation device (MicroPulser electroporator (Bio-Rad)).

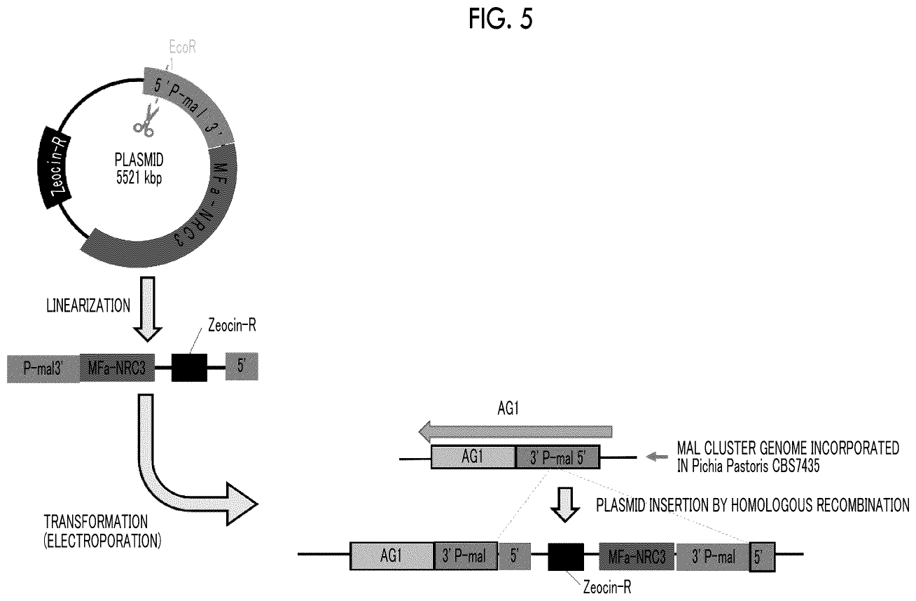

The Pichia pastoris CBS7435 strain transformed by electroporation was seeded on a YPD agar plate containing an antibiotic, zeocin (500 μg/mL) as a selection marker, and screening was performed to acquire a proliferated colony. As a result, as shown in FIG. 5, a transformed yeast in which an expression unit containing a MAL promoter and an NRC3 (CBE3) gene was incorporated into a sequence of the MAL promoter in the expression unit containing the MAL promoter (also referred to as P-mal) and an AG1 gene was acquired.

FIG. 5 shows a schematic diagram illustrating that the linearized plasmid is inserted into the AG1 site on the MAL cluster by homologous recombination.

### <Example 4> Culture and Protein Expression

To examine protein expression, the transformed yeast produced in Example 3 was cultured using a 3 L fermenter. 9 mL of the yeast culture solution obtained by the 24-hour expansion culture was collected, and 23 mL of Biotin and 5.1 mL of an antifoaming agent (struktol J673, Schill and Seilacher) were added to the batch medium 900 mL (Batch Medium 225 mL+water 675 mL) in the jar at the same time to start the fermentation. 24 hours after the start of the fermentation, 5.1 mL of an antifoaming agent (struktol J673, Schill and Seilacher) and a sucrose aqueous solution were added. 3.6 mL of TES was added at each of 24, 48, and 72 hours after the start of the fermentation. Ammonia was automatically added (cumulative amount of about 130 mL) by receiving feedback from a pH sensor, as a nitrogen source and for pH adjustment. A total of 970 mL of sucrose was added from 24 hours after the start of the fermentation to the end of the culture.

$$Sucrose addition rate : L ( mL / min ) = 0.262 \times {e}^{0.01 t} ( e is natural logarithm : 2.718 )$$

t is time (hr) from the start of the fermentation.

The pH was maintained at 5.5, and the pO2 was maintained at 25% from 2 hours after the start. The fermentation was stopped 100 hours after the start of the culture, and the culture solution containing the produced protein was recovered.

TABLE 1
Solution composition

| Name | Composition |
|---|---|
| Batch Medium | phosphoric acid 163.4 g/L |
| | sulfuric acid 2.5 mL/L |
| | potassium hydroxide 36.0 g/L |
| | TES 17.4 mL/L |
| | calcium chloride dihydrate 2.0 g/L |
| | magnesium sulfate heptahydrate 37.4 g/L |
| | glycerol 240.0 g/L |
| Aqueous ammonia | 25% to 28% (w/w) aqueous solution |
| Antifoaming agent | 10% Struktol J673 |
| Sucrose aqueous | 50% (w/w) aqueous solution |
| solution | |

The expression level of the expressed protein was evaluated from GPC analysis (equipment used: Waters e2695 HPLC alliance). The results are shown in the following table (column of sucrose-inducible promoter-introduced yeast in the following table).

As a comparative example, the measurement result of the expression level of expressed protein in a case of using the methanol-inducible promoter-introduced yeast is also shown in the following table (column of methanol-inducible promoter-introduced yeast in the following table).

### <Comparative Example 1> Production of Methanol-Inducible Promoter-Introduced Yeast

A plasmid vector in which a gene of a target protein (CBE3) and a zeocin resistance gene were linked downstream of a methanol-inducible AOX1 promoter was produced using a pPICZα plasmid vector (Invitrogen).

The nucleotide sequence of the produced plasmid vector is set forth in SEQ ID NO: 19.

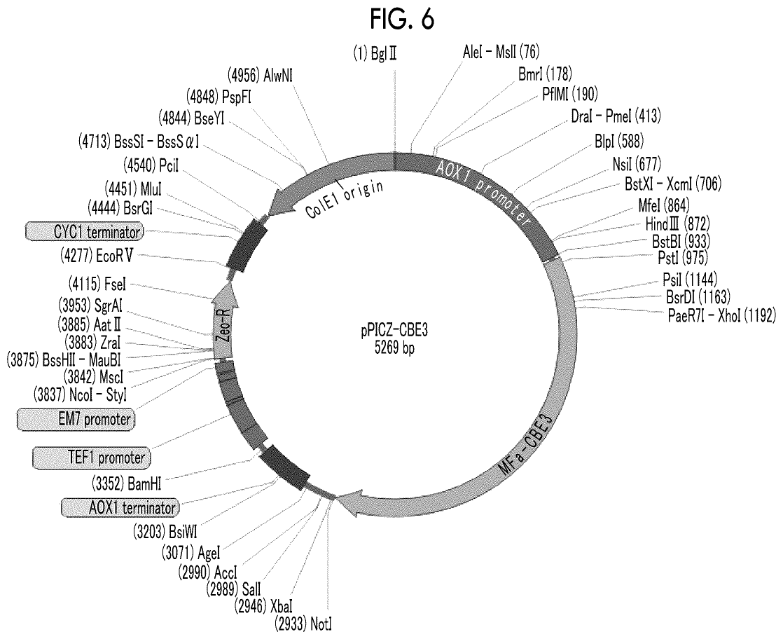

FIG. 6 shows a configuration of the produced plasmid vector.

The Pichia pastoris strain (X-33) transformed by electroporation was seeded on a YPD agar plate containing an antibiotic, zeocin (500 μg/mL) as a selection marker, and screening was performed to acquire a transformed yeast strain from a proliferated colony.

### <Comparative Example 2> Culture and Protein Expression

To examine protein expression of the methanol-inducible promoter-introduced yeast, the transformed yeast produced in Example 0090 was cultured using a 3 L fermenter in the same manner as in the sucrose-inducible promoter-introduced yeast. 8 mL of the yeast culture solution obtained by the 24-hour expansion culture was collected, and 23 mL of Biotin and 5.1 mL of an antifoaming agent (struktol J673, Schill and Seilacher) were added to the batch medium 900 mL (Batch Medium 225 mL+water 675 mL) in the jar at the same time to start the fermentation. 24 hours after the start of the fermentation, 5.1 mL of an antifoaming agent (struktol J673, Schill and Seilacher) and methanol were added. 3.6 mL of TES was added at each of 24, 48, and 72 hours after the start of the fermentation. Ammonia was automatically added (cumulative amount of about 100 mL) by receiving feedback from a pH sensor, as a nitrogen source and for pH adjustment. A total of 850 mL of methanol was added from 24 hours after the start of the fermentation to the end of the culture.

$$Methanol addition rate : L ( mL / min ) = 0.229 \times {e}^{0.01 t} ( e is natural logarithm : 2.718 )$$

t is time (hr) from the start of the fermentation.

It is noted that the methanol addition rate was set to be the same as the carbon equivalent per unit time of the sucrose addition rate.

TABLE 2

| | | | Expression |
|---|---|---|---|
| | Yeast wet | Survival | level of protein |
| Yeast | weight (g/L) | rate (%) | (g/L) |
| Sucrose-inducible | 640 | 100 | 6.8 |
| promoter- | | | |
| introduced yeast | | | |
| Methanol- | 117 | 8.8 | 0.05 |
| inducible | | | |
| promoter- | | | |
| introduced yeast | | | |

In the sucrose-inducible promoter-introduced yeast according to the embodiment of the present invention, a protein expression level higher than that of the conventional methanol-inducible promoter was obtained. In addition, in the methanol-inducible promoter-introduced yeast, the survival rate was decreased by adding methanol, but in the sucrose-inducible promoter-introduced yeast, there was no effect on the survival rate by adding sucrose, and continuous protein expression was suggested.

(SEQ ID NO: 1):
Sequence of MAL cluster
GTATTCCCGGAAAGAACTCGGGATACGGCCGGATTATTAGCTAAACGAGCCATGTTAGCA
AAACGGTTGGCGAGGTTTATTTTTTTATTTACATTTCACTGCAAGTCGATCGACCGTGTC
GCGACTAATGAATAATGAATATATTATGAAATGTCAAGATTCAACTAGTCTATCCCAATC
TGGCTCCGCAAACGCTTCGACTGTCTCATAAAACAAAGAAAATATTCAACTATATACCTT
TATTAGGCTGTAAAATCTAATTGACCTCGATCAGTCTACCCTCATATGGTTGCAGCTTGC
CTGTGATGTTGTTTGCAATATTAGACAGGAGCAGCTTTCCTTGAGGGATTGGGTACTCGA
CTTCTCTATCAGTAAAGTTCAAAACAACAGCCATCTTCTTTTGGCCTTTGTTGCTGGTCT
TGGTGTAGTAGAAAACCTCCTGGTTATCGTAGTCTTGCACCTCGAAACGGCCAAAAGTCA
ACGTCTCAGGATAAAGCTTTCTGGTCTTGATGGCATTTCTGTAAAAGTTGAGCACAGAGT
TAGGGTCATCTTTCTGCGAAGCAACGTTGATGTCTTTGTAGTTGTCGTTGATTCTCATCC
AAGGCTTACCACCGAAACCACCGTTTTCAGAAGAGTCCCACTGAACTGGAGTCCTTGCAT
TGTCTCTTGCCAGCAAATTGATGATCTTCAGCAGCTCGGCCTTCTCCTCATCAGAGTGCT
CGGTCTCGTTGAACGCGTTCCAGTAGTTAATGGTGTTGATATCCAAATACTCTGAAATAT
CCCATTTCGGTGAAACATTTGTCATACCAATCTCTTGACCTTGGTACACAAACAGAGTGC
CGGTCAAGGTTGTCTGCAACAGTGCCAGCATCTTTGCAGATCTGCTCCAAAACAGTTTGT
TGGAGGTGTTTCCGAACCTTGTGACACAGCGTGGTTGGTCGTGGTTCTCGATAAAAACGG
TGGACCATGCGTCATTCAGCTCACCGGTCTCGTCATCAAAGATGAATTCTGACTGGTTGA
TGATGGCATCCTTGAAGTCGGTCAAGTTGAAGCCTTTGTATCTAAAACGGTCGCTCTTGT
CGGATCCAACGTCAACAGTGTCGAACAGGAACATCATGTTCATCTCCTTTTCTTTTGCAG
AGACGTACTTGAGAGCATCTGCCTTGGAGCAGTGACCAACTTCTCCGACAGTCATGGCAT
CATACCTGGAAGTCACTTTCTCGTACATCTCTTTGTGGAACTCGTGAATACGTGGACCGG
AGTTGATGAGTGGACCAGCCGGCTGGTATTTCTCACCTGGGAAGGTGATTGGCGCATCCT
CAAAAGTCTGGACTTTTGAGTAGAGGCCAGCAGTGTCGATTCTGAAACCAGAAACGCCCT
TCTCGTACCAAAACTCAAGAGCAGATTTGTAAATGGCTTCACGGGTCTCTGGGTTCTCCC
AGTTCAAATCAGGCTGAGTCTCAGCAAACAGGCGAAGGTAGTATTGCTTAGTCTTTTCGT
CATAAGCCCATGCAGAACCAGAGAAAAAGGAAAGCCAGTTGTTGGCATTGTCCTTCCAAA
TGTACCAATCTCTCTTTGGATTGTCTCTGCTAGATCTAGACTCCTTGAACCAAGCGTGCT
CAGACGAGGTGTGGTTAATAACGAGGTCCAGAATCAATCTCATGTCTCTCTTCTTGATCT
CGGCAAGCAGTCTGTCCATGTCCTCCATAGTGCCAAATTGAGGGTTGATGGCATTGTAGT
CAGAAATATCGTATCCCATATCGTCCAATGGCGAAGCATAGTGCGGAGAAAGCCAGATCA
CATCAGTACCAAGGGATTTGATATGGTCGAGCTCGGAAGTGATTCCGTTTAAGTCTCCAA
TACCGTCTCCGTTGGAGTCTTTGAAGGAAGCAGGCCAAACTTGGTAAACAACGGCGGATT
TCCACCAAGGTTCTTGAGACTCGATAGTCATAAGGAATTAATATTGTCAAGAGGGATAAA
AAAATTGTGGAGGCGGAGGGTCTTTATATGGCTTGAGCTTACTAAAGACATTCTCATCAC
TTTAGCGAGCGCGACGCTTTGGTATCCTCCTAACTTCCCGAAAATAAAAAGAGGATTTCC
GTTTGCGTCATGGGCTCCACCTCCTATGCACGTCCATAATAGTTATTTGATTGTTATCTG
TACCCCAGACAAGATCCTGTTTCAGCGAGTATTAAATCACGGCCTAATTTATTTGAGCGA
GAATTAATTTGCACATTTAATTCTCGCTGAAATAACTGCCAACAATTGAGTCTGGGGTAC
ATGAATAGAAGTGACGTAATTGACCCCCACGCTTGCTAGCCACTGCGGAGTCACTTGAAC
CACATGAGAGCCCGTAGGGAAGCCAGAGCAATCAACGTCAAAGGGCTCTTAGGAAACTTG
TATAACCCAAGTGTTCATTACGACGCTGCAGAGCTCTCACATTATATGTAGAGTGGTACG
GCAGAGTCTGTAGAACATACAAAGCTATCATGTCGCGTAATCTGCACAGTGCCTGCTTTC
TATTGCGATACTCCCGAAGATATCCCTATCCGCATACACGGAACTGTAGTTAATTTGCCG
TGGAGTATCAATGTTTGTCCAACGCGCCGAAACTCTTTTTGCGGGGCTCTTCTTCATCTA
ACTCGTCCTAAGCTGGCTCGGGGTATCGCATAAACGTCTGCGTGCATTGCCAAGTTGTTT
TGGTCTGTGTTGAACCTTCTTTCCAAGCCCCATCCCGAACCCCGGAAAATTCCCCGATTA
TTATTTCCCCGCAAATGCGGCGTGCCAGAATCTCTCAGCCTTGCGTCATAATTTAGATTT
CGCAATTATTTCGTGGAGCAATAGGCCGCGAGCGTCCTATTGTGCTGTTGGCTCGAATCT
CATGCACAAAAATGAGGATTTGCCAGAGTGTGTTTTCCAAGGCGTGGCCAAAACAGAGGG
TCACCAGACAACAGAGCTCACCATAATACACAGCTTTATGGTCTTACACTAAGTCCTCCT
CCTCAACAACGCGGGGCTATACGTGTTTGCAGAATTCTTCCATGCGTGATTTGCAATGGT
GCTTGCATACTGCAACAATGCGTATGGGGTAATTAAAACGAGCACTCGCGAGCATTCATC
AAAGGTAAAAAGCATTATTCTAAGCTCTTTCTGGAAGCAAACTTGGGCCGCGATCACGCA
CCAAAATTCAGCCGGATTAAGAATGGGCAAGCATCTCCCCACGTCATATTTGTGTGGCAT
TAGGACTATATATAGTTGATATATTCTCGACACACTAGTATCCATCAGCATGCCTGAGTT
CGTCGAAAACATCGAGAAGCCTGAAGAAGTGGAAGTCATTCCAGACATCACAAAGAAAAT
TAACACTCTTTCTGACAGTGATGATGGAAGTGGTGCTTTCAACGACTACATTGCCAGATT
CGTGGAGATTTCGACGAATGCCCAGAATAATGAGCATCAAGAAAAACACATGTCGCTGAA
GGAAGGTCTGAAAACTTTCCCGAAAGCTGCCTGCTGGTCGATTGTGCTTTCCACAGCCAT
CATCATGGAAGGATACGATACCATGCTCTTGAATAGTTTGTACTCGATGCAATCTTTCGC
CAAAAAATACGGCCAGTACTACCCGGAAATTGACGAGTACCAGGTCCCTGCCAAGTGGCA
GACGTCGCTTTCGATGTCGACTTATGTCGGTGAAATTGTCGGACTGTACATTGCAGGTCT
TGTTGCCGAGAAATGGGGATACAGACGTACGTTGATATGCTTCATGGCAGCCGTTGTGGG
TTTAATCTTCATTCTTTTCTTTGCGGTCGACGTGCAGATGCTGCTTGCCGGCGAGCTGCT
CTGTGGTATTGTCTGGGGTGCATTCCAGACTCTTACAGTGTCATACGCCTCTGAAGTCTG
TCCCGTTGTCCTGAGAATCTACCTTACCACTTATGTCAATGCGTGCTGGGTTATCGGACA
GTTAATTGCTGCTTGTTTGCTGAGAGGCACTATGACCCTGACCAACGAGTGGTCATACAA
AATTCCTTTCGCCGTTCAGTGGATCTGGCCTGTGCCAATCATGATTGGAATCTACCTGGC
CCCGGAGTCGCCCTGGTGGCTGGTTAAAAAGAACCGGGACGCAGAAGCCAAGAGAAGTAT
CATGAGACTTTTGAGTCCAAACACCGAGGTGCCAGACGTTGCTCCGCTAGCCGAGGCAAT
GCTGAACAAAATGCAACTAACCATCAAGGAAGAGTCTGCGCGCACATCCAATGTCTCGTA
CCTTGACTGCTTCAAACACGGCAACTTCAGAAGAACAAGGATTGCCTCCATGATCTGGCT
CATCCAGAATATCACTGGATCTGTCTTGATGGGCTATTCGACCTACTTTTACATCCAGGC
AGGACTTGATAGCGGTATGTCTTTCACTTTCTCTATCATCCAATATGCACTGGGTCTTCT
TGGAACAATTGCCTCGTGGCTGCTGTCGCAAAAAATGGGCCGTTTCGACATCTATTTCTT
GGGTCTTAGCATAAACACATGCATCCTGATAATTGTCGGAGGTTTGGGCTTCTCTTCATC
GAGCAGTGCGTCTTGGGCAATTGGTTCGTTGCTCCTTGTCTTCACTTTTGTCTACGACTC
ATCCATCGGTCCTATCACCTACTGTACGGTTGCAGAAATCCCTTCGTCAACGGTGCGTGC
CAAGACAGTTGCCCTTGCAAGAAACTGGTACAATCTGTCTCAAATCCCGCTCTCCATTGT
CACTCCATACATGCTGAATCCGACTGCTTGGAACTGGAAAGCAAAAGCAGCCCTTTTATG
GGCAGGCTTGTCTGTTTGCTCACTGATCTACATCTGGTTCGAGTTCCCAGAAACAAAAGG
AAGAACTTATGCTGAACTGGACATCCTATTCAAAAACGGTACCAGTGCCCGTAAGTTTAA
GTCCACTCAGGTGGAAACATTCAATCCTGAGGAAATGTTAAAAAAAATGAATAATGAGGA
TATTATACAGGTTGTTGATGGCGACCTGGATGCCGGTGCGGCCACTGCTAAAGTTTAAAA
GTTGAAATGTATAGTTTCAGTTCTGCGATGTTATTATTTTACAATTGAATATGCTATCTC
GAATGATTTTTTGTAATAGCCGTAGATGTCTTTATTATTGCATTTAAGGGTTCAAAATTA
TTCATGTGAGCGTTTTATTTTACGGGACAATAAGATTTTGGGCGAGACTTCCCCACAAAT
TGAATAATGTGCAATATTTTTAAGAACTAAATTTACTGGAGACAATTTCTATGCCTCTCA
GTACACGTGCAAAACCACAAACGCCTGGCCAAAAGTCCCATTCTAAAAGGCCGTATATCA
GGCCTTGTGACGCGTGCGCGTTCAAAAGAGTACGTTGCGATGCTGAGTTCAAGTTGGATC
GCAGATGCACCAACTGTTTAGTTCATGGAATTGAGTGTACGAATAACAGAGTCAAGCAGA
GATCTGGACCCAGAAAAATACATAAAAAAACTGAAGAGGCTATTAAAAGCCTGGTAGAAG
GAAGTGGCGCGTTCAGCAATCTTCGATTAGCCACTCCGGCCCACAACATATGTGGTATTA
TCAATCCTGCCACCTGCGAGAAGAGCTCAATATCGCTTAGTGAGATTCGACCGTACCTCA
GAATTTATAAATCCCGATACTATGCACTCTGGCCAGTCCTGTCTGTTGATGACTGTCTGA
ATATTCTTAATGAAAGCTCAGTTCACATAAACGATAGCGCATTTTTCATGCTCAATGCCA
ACAATGCGTCGTTGTACGCTCTTTCATGCGCCTTGTGCGCTGTAATTGCAAGACACACCA
CCTTCTGGTCCCTGGAAGAATTTGATACAATGGCTCATTTAGGACTCAAAAGCTGCCCTC
CGCCAGAAACTTATGTAAGGGAGGCAGAGCGAGCTATCTACATGTTTGACCTTGACGGGA
TACTCACAGAAGACCAGGTTTTAACTAACTTCTTTCTGCATATATATTTTTCATCCCTTG
ACGAGGACAACCTGCAGGAATTGGTCTATCTCAGACAGGCAATCAGCTGCGCACAAATGC
TTCACCTTCGAGATAACGAAGTTTTGGGAGAATCACCAAAAGATACATCACACAGATTTA
AAAAAATTTACTACTTACTTCTCATCACAGAGAGGTATGTCTCGTTCAAAAAACAACTAC
CAGTGATATTAGAGCCAACAATGCCACTTCCAAGTTTGGAAGATGATGAAGCCCCGGATC
TGCTACCTGGATTTATCGAACTTGCGCAAGTATTCAGTGTTCCTGATTGCGCTTTCTTTA
ACAAGTACTCGCTCCGTGGCACGACAGCTGCACTTGATCCTTTCAGCGAGAGCTTTGACC
TAATGTTCAAAAACGAATGGATCCAAGACGTGCAAACCAAGCTCTCTAGGTCGGTCGTTC
AGGTGCGCAACCAGTCTCAAAAGGTCAATATATTAATTTCTCGAACATGGTTGCAATCAA
TTGCTTGGTTAATGGCCAAAGATCGACTTCTTCTATCGCCTACGAGTGATCAAGCCAATT
TCTTTGATCCAAGGTATCCTATTCATATCGCGAAAGACTTTCTGATACAGACTAAAGACA
TGCCGTACGAGGCATTTGAAACAAACGGTAGTGGTGTCATCGTGAAACTAAGCGAAGTCG
CCAACGCCTTGCAAACCTTCATCCGATTGGCTCCAGGATCGCCCGAAACAGCTTTAGCAT
TAGACGAGCTTGCATATATCCACGGTATTATCATGCGGCAGAACTCCAACAGCTCGCTCG
GGCCTCTTATACACCCTATCACTGAAACTTTGGAATCTCGCAGGTTCGGATTTTATCGAA
ATGGCTGCGACATTTCTTATCCAAAAATAGAGTCTGCAGATTATGAAGAGAACGAAAATG
ACCATACACCCGAAATCATTGCCTCCATGCCGGAGAACTTGAATTTGACACCTCTTTTGA
GCGAGTTCGAATTTCCGAGGTGAACAAGTATTATCTGCTCACGTGTTTGGTGCATTTTGC
AACTAGCGACAAATTTCACTGGCATATTAGTATATCATCAAATCATTAGGATTGCTATTT
TGTGTCCACTAAGGCGCTCCAGGTGCGTGGAATGGTGCAGTATTAGGCCCTAGCTGTCCG
AGAAAAAAGGAAGCTACACCCTTTGATGTAGTGGGCTCTTTGTGTGTTGTGAGAGGATTA
AATTGGCCAGATGACACTCTTTCCAAAGATGCAAATAACGAGGAAGAAACATCCAGCCGG
ATGCCAGATGTCTGTCTGTGTTGCAAATTCGCTCTTGATCCGGAAAAAGAGTTATTGAAA
AGGGCACTGTCCTGTCTCGTATTGCAGCGTACGAGTGAACCTGAGCGTGTCAATGCAGGT
TCATAAGAAGAAAATTCAGAAGACAGATTGTGACATATTTCTCGCAAGCCATTCACTTTC
CAAGATTCTAGGAGCTTAGAGATCTTGGTGAACAGGGCTCGGGGCAAGTCCATTCCAGCT
TTGTACTTCAACACCATGTCGAATACGGCACCCATGCGTTCGTAGGCTAGCTCGGTGGTT
GCTGGGTCAACATAGCTTTTGATCAGAATGTAAAGAGAGTCTGCTATCTCCAGTAATTTC
ATTGATATGTCGGGTCCGTTGCATCTAAAGGCATGATCAGGGAGGTTTTGTAGACTGCTG
AGAAGCTCATTTGCAACTTTTAGAGGAAAGTTAATCTTGAAACATTCGTGTTCGACCGCA
ATGCTCTCATCAGAACTAATTAGAAAATTCGCAGACGCAATTAACCAGCCCAAAGACTGG
AGCCAGGCTTTTGAGACAATAACATTCACTTTTTGAGCACTTGTTGCAATTTTATCCACA
TCAATTCTAATTTGGAGCTTCTGTTGGAGCTCAAGGAGCCACAGCCTCTTCAACTCTGTG
GATCCGTTCAAGGTAAAATCCTGAAACAAGTTGGTCTCTGGAATCTGGAACATGTTGTGA
GTGCGCCCTGTCTCGAGCACTTTAGTATGAAGCTCAACATAGAATTGCTTATCCGTTGCA
CTGAAAATTGCACACAGAGTGGTGAACCCGTAAAGTGAATTTGAGGATTTTTCGAAATCT
GCTTCAGGGAGTGGAATTGAAGGTTCAAGAAGAACGGGCAAATTAGTTTTAAAGCTGACA
AACCTTTCGGTTACCACGAGCAAATAGTAGATTTTTTTCTGCCTATGAGCCTCAACGGGT
GACAAATGAAGCAGTGACTGTGGTTCGTGCATTTTTAAGATATCTGCAAGAGAAACAGCT
TCACGGAGGTAAATAAGACCTTTGTGCTGTTGGCCAGGCGCAATAGCATAGTATTTATGT
AGGAAGAAGGATGTCAGAAGGAGATCAATATTTGGTGTGCTTTTCAGTCTATACTCATAA
ATTATCCTCCTTGCCTCTTGAGCATATAAGCCAGCATTTTTGAGATCGCTCCTGGCTGGA
GTGTATGTGACGAAACTGTGATATTGTGCGATCGTAGCGCACAATGCGCAACAAAGAGCA
TATTCTTGGGCATTGTCCTCGGCGATGGTGATACGAAAATCATCTAAAACATTCGCTAGT
TGAAATACCTGCTCCACATTCTTTAAGGGGCTTGAACGTGTCAAAATTGAAACTGTACTT
TGAATTGATACTACCGGCCAGATATCGTAAAAATTCGAATGATATATCTCAAGGTAAGGG
ATAATTTGTTTCAAAGAAAATGCGCACACATGAGCAGGTAGTGTAGACTGAGCATAGATT
TCGTCGTTGAGATCCTCTGTGTTTTTGACTGTTCTAGTACTTTTCTTTGGACCGGATTTA
TGCCTCACGCGGAGGTTGGTACATGGAACTTTATGAGTGAGACAGTTCGAGCAAATACCA
CCGGTGCATGAATCTATATCGCACTTCACACGGCGGATGGCGCATGCGTCACAGGGTCTC
GGCCGTTGTCGTTTTTTGGTGGACGGCTGCGACATCTCGGTATTTAGATAAGCCAGCTTG
TACTTGTATCCAAAATGTGGGGCTAAAGGGGAGTGTATGATTTTTAATGATATCGGTGAA
TGTCCAGAAATTTGGTAAATCGCACACCAAACTTCAACGATGGACTAGCGTGCTAAACTG
ATATTCTACCAAACAACCGTTTGAGTAATTCTGACCATGACAGAGGTCTTGTGTATATAT
TTTAAGCCCCGTCGATCGACCGCATCGCGACTCCATAAAAAAATTCATTTCACGCGTTAT
TATTATCGTATGAATATGGATCCCCTAAACGTGAAAGTGCAACAGAAACTTAAGGAGCTC
GAAAGCCTTCAGCAGATAAGAGACTTGACGAAACACCTGAATGTTTCACTGGAAGAATTT
GCTGGCCAAATAGAACTTCTGGGTGAGGAAGCAGGATGCATCGAAACCGTGACGCAAAAC
TGGATGAGAATCATCAGAGCAGTGAGCCTAGCCTCTAACTCTTTGTCGAACTACAAGGAA
GAGGACTATGAAACGGATAGACCAATGACGGAGCGCCTTGTGAGATGCAAGATAGACGAG
AGTCAAAAAATCATCACCAAAAATTGACTTACTTCAATCTATCTAACCATTGTTCTAAAT
TGATCTTGTTGTTCAGTTCCGACAACAGATCGTTCTCCTTCAGCAAATCGTCACGAAGGC
CTTTATCAGCCTCGAGATAATTAATATCGTCCGGATCTAACATTAAGTCCTGAACTTTAG
AGGGAAGTTCTATTTTGAGCGTCGATAACCCGGTCATCATTTTGAAGTATTCATTAGTAT
GCACGTCAGAACCATTCAAAAGCCCATCCCAATCAGTTGTCAAAAACAGCCTTTTCCATA
TCAAGGTTTCCAAGATTTCGTGATAGTGCGAATAACCACTCAGGTTACTTATCACTCTTG
ACGGATTGGAGTGTTTGATGACAGTCAACAATTTGAACAAGTTGGCGAAATTGTTGTGAC
TGAATTCATCAAACGTCACTAATGTCAGTAGCTGGACCATCTCAAACAATCCCAGCTGTT
GCTGCTTATCCATCTTTGCCACAATATCGCCGATCTGACTTTCAAAATGTGTAAACTTGT
TATCAACCAAGTATCTTGAGTCTTGTATGAATCCATCTTTAGTTTTTGAATCAATTAATC
TCGCATAATTCAATTGAAGCTCAATCATTTGCAAGTGTATTTCTATCTCATCCAACAGAT
TGTCGTGATCCTCATTCAAAGCTATCAGTGAAAGTTTGGCAATATTCAGTTCGAACTTTT
TCTTTTCAATTTCCTCCGACTCTGCAGTGGAAACATATGCAATCAGCTTTTCTGCTGCCT
TGAAATACTCATCGTTTTCCACGTCAAGTATCCAGGCAAATTTATAGTTGTTGATGCTTG
AATTCAAAAACATGCGCACGTAATCTGGATATTGAGGGATGTAATTGAGGATTAGCCGAA
TCTTTCTGGTTCTAGCATAGAATCCAAAAAGCACTTCTGCAAAACGGTATCCATACCGCT
GGAAACATTCC
(SEQ ID NO: 2) (fragment amplified with KP-MAL3 and KP-MAL4)
CAAATCAACTGAGAAAAATGGTATTCCCGGAAAGAACTCGGGATACGGCCGGATTATTAG
CTAAACGAGCCATGTTAGCAAAACGGTTGGCGAGGTTTATTTTTTTATTTACATTTCACT
GCAAGTCGATCGACCGTGTCGCGACTAATGAATAATGAATATATTATGAAATGTCAAGAT
TCAACTAGTCTATCCCAATCTGGCTCCGCAAACGCTTCGACTGTCTCATAAAACAAAGAA
AATATTCAACTATATACCTTTATTAGGCTGTAAAATCTAATTGACCTCGATCAGTCTACC
CTCATATGGTTGCAGCTTGCCTGTGATGTTGTTTGCAATATTAGACAGGAGCAGCTTTCC
TTGAGGGATTGGGTACTCGACTTCTCTATCAGTAAAGTTCAAAACAACAGCCATCTTCTT
TTGGCCTTTGTTGCTGGTCTTGGTGTAGTAGAAAACCTCCTGGTTATCGTAGTCTTGCAC
CTCGAAACGGCCAAAAGTCAACGTCTCAGGATAAAGCTTTCTGGTCTTGATGGCATTTCT
GTAAAAGTTGAGCACAGAGTTAGGGTCATCTTTCTGCGAAGCAACGTTGATGTCTTTGTA
GTTGTCGTTGATTCTCATCCAAGGCTTACCACCGAAACCACCGTTTTCAGAAGAGTCCCA
CTGAACTGGAGTCCTTGCATTGTCTCTTGCCAGCAAATTGATGATCTTCAGCAGCTCGGC
CTTCTCCTCATCAGAGTGCTCGGTCTCGTTGAACGCGTTCCAGTAGTTAATGGTGTTGAT
ATCCAAATACTCTGAAATATCCCATTTCGGTGAAACATTTGTCATACCAATCTCTTGACC
TTGGTACACAAACAGAGTGCCGGTCAAGGTTGTCTGCAACAGTGCCAGCATCTTTGCAGA
TCTGCTCCAAAACAGTTTGTTGGAGGTGTTTCCGAACCTTGTGACACAGCGTGGTTGGTC
GTGGTTCTCGATAAAAACGGTGGACCATGCGTCATTCAGCTCACCGGTCTCGTCATCAAA
GATGAATTCTGACTGGTTGATGATGGCATCCTTGAAGTCGGTCAAGTTGAAGCCTTTGTA
TCTAAAACGGTCGCTCTTGTCGGATCCAACGTCAACAGTGTCGAACAGGAACATCATGTT
CATCTCCTTTTCTTTTGCAGAGACGTACTTGAGAGCATCTGCCTTGGAGCAGTGACCAAC
TTCTCCGACAGTCATGGCATCATACCTGGAAGTCACTTTCTCGTACATCTCTTTGTGGAA
CTCGTGAATACGTGGACCGGAGTTGATGAGTGGACCAGCCGGCTGGTATTTCTCACCTGG
GAAGGTGATTGGCGCATCCTCAAAAGTCTGGACTTTTGAGTAGAGGCCAGCAGTGTCGAT
TCTGAAACCAGAAACGCCCTTCTCGTACCAAAACTCAAGAGCAGATTTGTAAATGGCTTC
ACGGGTCTCTGGGTTCTCCCAGTTCAAATCAGGCTGAGTCTCAGCAAACAGGCGAAGGTA
GTATTGCTTAGTCTTTTCGTCATAAGCCCATGCAGAACCAGAGAAAAAGGAAAGCCAGTT
GTTGGCATTGTCCTTCCAAATGTACCAATCTCTCTTTGGATTGTCTCTGCTAGATCTAGA
CTCCTTGAACCAAGCGTGCTCAGACGAGGTGTGGTTAATAACGAGGTCCAGAATCAATCT
CATGTCTCTCTTCTTGATCTCGGCAAGCAGTCTGTCCATGTCCTCCATAGTGCCAAATTG
AGGGTTGATGGCATTGTAGTCAGAAATATCGTATCCCATATCGTCCAATGGCGAAGCATA
GTGCGGAGAAAGCCAGATCACATCAGTACCAAGGGATTTGATATGGTCGAGCTCGGAAGT
GATTCCGTTTAAGTCTCCAATACCGTCTCCGTTGGAGTCTTTGAAGGAAGCAGGCCAAAC
TTGGTAAACAACGGCGGATTTCCACCAAGGTTCTTGAGACTCGATAGTCATAAGGAATTA
ATATTGTCAAGAGGGATAAAAAAATTGTGGAGGCGGAGGGTCTTTATATGGCTTGAGCTT
ACTAAAGACATTCTCATCACTTTAGCGAGCGCGACGCTTTGGTATCCTCCTAACTTCCCG
AAAATAAAAAGAGGATTTCCGTTTGCGTCATGGGCTCCACCTCCTATGCACGTCCATAAT
AGTTATTTGATTGTTATCTGTACCCCAGACAAGATCCTGTTTCAGCGAGTATTAAATCAC
GGCCTAATTTATTTGAGCGAGAATTAATTTGCACATTTAATTCTCGCTGAAATAACTGCC
AACAATTGAGTCTGGGGTACATGAATAGAAGTGACGTAATTGACCCCCACGCTTGCTAGC
CACTGCGGAGTCACTTGAACCACATGAGAGCCCGTAGGGAAGCCAGAGCAATCAACGTCA
AAGGGCTCTTAGGAAACTTGTATAACCCAAGTGTTCATTACGACGCTGCAGAGCTCTCAC
ATTATATGTAGAGTGGTACGGCAGAGTCTGTAGAACATACAAAGCTATCATGTCGCGTAA
TCTGCACAGTGCCTGCTTTCTATTGCGATACTCCCGAAGATATCCCTATCCGCATACACG
GAACTGTAGTTAATTTGCCGTGGAGTATCAATGTTTGTCCAACGCGCCGAAACTCTTTTT
GCGGGGCTCTTCTTCATCTAACTCGTCCTAAGCTGGCTCGGGGTATCGCATAAACGTCTG
CGTGCATTGCCAAGTTGTTTTGGTCTGTGTTGAACCTTCTTTCCAAGCCCCATCCCGAAC
CCCGGAAAATTCCCCGATTATTATTTCCCCGCAAATGCGGCGTGCCAGAATCTCTCAGCC
TTGCGTCATAATTTAGATTTCGCAATTATTTCGTGGAGCAATAGGCCGCGAGCGTCCTAT
TGTGCTGTTGGCTCGAATCTCATGCACAAAAATGAGGATTTGCCAGAGTGTGTTTTCCAA
GGCGTGGCCAAAACAGAGGGTCACCAGACAACAGAGCTCACCATAATACACAGCTTTATG
GTCTTACACTAAGTCCTCCTCCTCAACAACGCGGGGCTATACGTGTTTGCAGAATTCTTC
CATGCGTGATTTGCAATGGTGCTTGCATACTGCAACAATGCGTATGGGGTAATTAAAACG
AGCACTCGCGAGCATTCATCAAAGGTAAAAAGCATTATTCTAAGCTCTTTCTGGAAGCAA
ACTTGGGCCGCGATCACGCACCAAAATTCAGCCGGATTAAGAATGGGCAAGCATCTCCCC
ACGTCATATTTGTGTGGCATTAGGACTATATATAGTTGATATATTCTCGACACACTAGTA
TCCATCAGCATGCCTGAGTTCGTCGAAAACATCGAGAAGCCTGAAGAAGTGGAAGTCATT
CCAGACATCACAAAGAAAATTAACACTCTTTCTGACAGTGATGATGGAAGTGGTGCTTTC
AACGACTACATTGCCAGATTCGTGGAGATTTCGACGAATGCCCAGAATAATGAGCATCAA
GAAAAACACATGTCGCTGAAGGAAGGTCTGAAAACTTTCCCGAAAGCTGCCTGCTGGTCG
ATTGTGCTTTCCACAGCCATCATCATGGAAGGATACGATACCATGCTCTTGAATAGTTTG
TACTCGATGCAATCTTTCGCCAAAAAATACGGCCAGTACTACCCGGAAATTGACGAGTAC
CAGGTCCCTGCCAAGTGGCAGACGTCGCTTTCGATGTCGACTTATGTCGGTGAAATTGTC
GGACTGTACATTGCAGGTCTTGTTGCCGAGAAATGGGGATACAGACGTACGTTGATATGC
TTCATGGCAGCCGTTGTGGGTTTAATCTTCATTCTTTTCTTTGCGGTCGACGTGCAGATG
CTGCTTGCCGGCGAGCTGCTCTGTGGTATTGTCTGGGGTGCATTCCAGACTCTTACAGTG
TCATACGCCTCTGAAGTCTGTCCCGTTGTCCTGAGAATCTACCTTACCACTTATGTCAAT
GCGTGCTGGGTTATCGGACAGTTAATTGCTGCTTGTTTGCTGAGAGGCACTATGACCCTG
ACCAACGAGTGGTCATACAAAATTCCTTTCGCCGTTCAGTGGATCTGGCCTGTGCCAATC
ATGATTGGAATCTACCTGGCCCCGGAGTCGCCCTGGTGGCTGGTTAAAAAGAACCGGGAC
GCAGAAGCCAAGAGAAGTATCATGAGACTTTTGAGTCCAAACACCGAGGTGCCAGACGTT
GCTCCGCTAGCCGAGGCAATGCTGAACAAAATGCAACTAACCATCAAGGAAGAGTCTGCG
CGCACATCCAATGTCTCGTACCTTGACTGCTTCAAACACGGCAACTTCAGAAGAACAAGG
ATTGCCTCCATGATCTGGCTCATCCAGAATATCACTGGATCTGTCTTGATGGGCTATTCG
ACCTACTTTTACATCCAGGCAGGACTTGATAGCGGTATGTCTTTCACTTTCTCTATCATC
CAATATGCACTGGGTCTTCTTGGAACAATTGCCTCGTGGCTGCTGTCGCAAAAAATGGGC
CGTTTCGACATCTATTTCTTGGGTCTTAGCATAAACACATGCATCCTGATAATTGTCGGA
GGTTTGGGCTTCTCTTCATCGAGCAGTGCGTCTTGGGCAATTGGTTCGTTGCTCCTTGTC
TTCACTTTTGTCTACGACTCATCCATCGGTCCTATCACCTACTGTACGGTTGCAGAAATC
CCTTCGTCAACGGTGCGTGCCAAGACAGTTGCCCTTGCAAGAAACTGGTACAATCTGTCT
CAAATCCCGCTCTCCATTGTCACTCCATACATGCTGAATCCGACTGCTTGGAACTGGAAA
GCAAAAGCAGCCCTTTTATGGGCAGGCTTGTCTGTTTGCTCACTGATCTACATCTGGTTC
GAGTTCCCAGAAACAAAAGGAAGAACTTATGCTGAACTGGACATCCTATTCAAAAACGGT
ACCAGTGCCCGTAAGTTTAAGTCCACTCAGGTGGAAACATTCAATCCTGAGGAAATGTTA
AAAAAAATGAATAATGAGGATATTATACAGGTTGTTGATGGCGACCTGGATGCCGGTGCG
GCCACTGCTAAAGTTTAAAAGTTGAAATGTATAGTTTCAGTTCTGCGATGTTATTATTTT
ACAATTGAATATGCTATCTCGAATGATTTTTTGTAATAGCCGTAGATGTCTTTATTATTG
CATTTAAGGGTTCAAAATTATTCATGTGAGCGTTTTATTTTACGGGACAATAAGATTTTG
GGCGAGACTTCCCCACAAATTGAATAATGTGCAATATTTTTAAGAACTAAATTTACTGGA
GACAATTTCTATGCCTCTCAGTACACGTGCAAAACCACAAACGCCTGGCCAAAAGTCCCA
TTCTAAAAGGCCGTATATCAGGCCTTGTGACGCGTGCGCGTTCAAAAGAGTACGTTGCGA
TGCTGAGTTCAAGTTGGATCGCAGATGCACCAACTGTTTAGTTCATGGAATTGAGTGTAC
GAATAACAGAGTCAAGCAGAGATCTGGACCCAGAAAAATACATAAAAAAACTGAAGAGGC
TATTAAAAGCCTGGTAGAAGGAAGTGGCGCGTTCAGCAATCTTCGATTAGCCACTCCGGC
CCACAACATATGTGGTATTATCAATCCTGCCACCTGCGAGAAGAGCTCAATATCGCTTAG
TGAGATTCGACCGTACCTCAGAATTTATAAATCCCGATACTATGCACTCTGGCCAGTCCT
GTCTGTTGATGACTGTCTGAATATTCTTAATGAAAGCTCAGTTCACATAAACGATAGCGC
ATTTTTCATGCTCAATGCCAACAATGCGTCGTTGTACGCTCTTTCATGCGCCTTGTGCGC
TGTAATTGCAAGACACACCACCTTCTGGTCCCTGGAAGAATTTGATACAATGGCTCATTT
AGGACTCAAAAGCTGCCCTCCGCCAGAAACTTATGTAAGGGAGGCAGAGCGAGCTATCTA
CATGTTTGACCTTGACGGGATACTCACAGAAGACCAGG
(SEQ ID NO: 3) (fragment amplified with KP-MAL1 and KP-MAL2)
TTGTCAGCTTAAAGGACTCCATTTCCTAAAATTTCAAGCAGTCCTCTCAACTAAATTTTT
TTCCATTCCTCTGCACCCAGCCCTCTTCATCAACCGTCCAGCCTTCTCAAAAGTCCAATG
TAAGTAGCCTGCAAATTCAGGTTACAACCCCTCAATTTTCCATCCAAGGGCGATCCTTAC
AAAGTTAATATCGAACAGCAGAGACTAAGCGAGTCATCATCACCACCCAACGATGGTGAA
AAACTTTAAGCATAGATTGATGGAGGGTGTATGGCACTTGGCGGCTGCATTAGAGTTTGA
AACTATGGGGTAATACATCACATCCGGAACTGATCCGACTCCGAGATCATATGCAAAGCA
CGTGATGTACCCCGTAAACTGCTCGGATTATCGTTGCAATTCATCGTCTTAAACAGTACA
AGAAACTTTATTCATGGGTCATTGGACTCTGATGAGGGGCACATTTCCCCAATGATTTTT
TGGGAAAGAAAGCCGTAAGAGGACAGTTAAGCGAAAGAGACAAGACAACGAACAGCAAAA
GTGACAGCTGTCAGCTACCTAGTGGACAGTTGGGAGTTTCCAATTGGTTGGTTTTGAATT
TTTACCCATGTTGAGTTGTCCTTGCTTCTCCTTGCAAACAATGCAAGTTGATAAGACATC
ACCTTCCAAGATAGGCTATTTTTGTCGCATAAATTTTTGTCTCGGAGTGAAAACCCCTTT
TATGTGAACAGATTACAGAAGCGTCCTACCCTTCACCGGTTGAGATGGGGAGAAAATTAA
GCGATGAGGAGACGATTATTGGTATAAAAGAAGCAACCAAAATCCCTTATTGTCCTTTTC
TGATCAGCATCAAAGAATATTGTCTTAAAACGGGCTTTTAACTACATTGTTCTTACACAT
TGCAAACCTCTTCCTTCTATTTCGGATCAACTGTATTGACTACATTGATCTTTTTTAACG
AAGTTTACGACTTACTAAATCCCCACAAACAAATCAACTGAGAAAAATGGTATTCCCGGA
A
(SEQ ID NO: 4) (fragment amplified with KP-MAL5 and KP-MAL6)
TGGGCAGGCTTGTCTGTTTGCTCACTGATCTACATCTGGTTCGAGTTCCCAGAAACAAAA
GGAAGAACTTATGCTGAACTGGACATCCTATTCAAAAACGGTACCAGTGCCCGTAAGTTT
AAGTCCACTCAGGTGGAAACATTCAATCCTGAGGAAATGTTAAAAAAAATGAATAATGAG
GATATTATACAGGTTGTTGATGGCGACCTGGATGCCGGTGCGGCCACTGCTAAAGTTTAA
AAGTTGAAATGTATAGTTTCAGTTCTGCGATGTTATTATTTTACAATTGAATATGCTATC
TCGAATGATTTTTTGTAATAGCCGTAGATGTCTTTATTATTGCATTTAAGGGTTCAAAAT
TATTCATGTGAGCGTTTTATTTTACGGGACAATAAGATTTTGGGCGAGACTTCCCCACAA
ATTGAATAATGTGCAATATTTTTAAGAACTAAATTTACTGGAGACAATTTCTATGCCTCT
CAGTACACGTGCAAAACCACAAACGCCTGGCCAAAAGTCCCATTCTAAAAGGCCGTATAT
CAGGCCTTGTGACGCGTGCGCGTTCAAAAGAGTACGTTGCGATGCTGAGTTCAAGTTGGA
TCGCAGATGCACCAACTGTTTAGTTCATGGAATTGAGTGTACGAATAACAGAGTCAAGCA
GAGATCTGGACCCAGAAAAATACATAAAAAAACTGAAGAGGCTATTAAAAGCCTGGTAGA
AGGAAGTGGCGCGTTCAGCAATCTTCGATTAGCCACTCCGGCCCACAACATATGTGGTAT
TATCAATCCTGCCACCTGCGAGAAGAGCTCAATATCGCTTAGTGAGATTCGACCGTACCT
CAGAATTTATAAATCCCGATACTATGCACTCTGGCCAGTCCTGTCTGTTGATGACTGTCT
GAATATTCTTAATGAAAGCTCAGTTCACATAAACGATAGCGCATTTTTCATGCTCAATGC
CAACAATGCGTCGTTGTACGCTCTTTCATGCGCCTTGTGCGCTGTAATTGCAAGACACAC
CACCTTCTGGTCCCTGGAAGAATTTGATACAATGGCTCATTTAGGACTCAAAAGCTGCCC
TCCGCCAGAAACTTATGTAAGGGAGGCAGAGCGAGCTATCTACATGTTTGACCTTGACGG
GATACTCACAGAAGACCAGGTTTTAACTAACTTCTTTCTGCATATATATTTTTCATCCCT
TGACGAGGACAACCTGCAGGAATTGGTCTATCTCAGACAGGCAATCAGCTGCGCACAAAT
GCTTCACCTTCGAGATAACGAAGTTTTGGGAGAATCACCAAAAGATACATCACACAGATT
TAAAAAAATTTACTACTTACTTCTCATCACAGAGAGGTATGTCTCGTTCAAAAAACAACT
ACCAGTGATATTAGAGCCAACAATGCCACTTCCAAGTTTGGAAGATGATGAAGCCCCGGA
TCTGCTACCTGGATTTATCGAACTTGCGCAAGTATTCAGTGTTCCTGATTGCGCTTTCTT
TAACAAGTACTCGCTCCGTGGCACGACAGCTGCACTTGATCCTTTCAGCGAGAGCTTTGA
CCTAATGTTCAAAAACGAATGGATCCAAGACGTGCAAACCAAGCTCTCTAGGTCGGTCGT
TCAGGTGCGCAACCAGTCTCAAAAGGTCAATATATTAATTTCTCGAACATGGTTGCAATC
AATTGCTTGGTTAATGGCCAAAGATCGACTTCTTCTATCGCCTACGAGTGATCAAGCCAA
TTTCTTTGATCCAAGGTATCCTATTCATATCGCGAAAGACTTTCTGATACAGACTAAAGA
CATGCCGTACGAGGCATTTGAAACAAACGGTAGTGGTGTCATCGTGAAACTAAGCGAAGT
CGCCAACGCCTTGCAAACCTTCATCCGATTGGCTCCAGGATCGCCCGAAACAGCTTTAGC
ATTAGACGAGCTTGCATATATCCACGGTATTATCATGCGGCAGAACTCCAACAGCTCGCT
CGGGCCTCTTATACACCCTATCACTGAAACTTTGGAATCTCGCAGGTTCGGATTTTATCG
AAATGGCTGCGACATTTCTTATCCAAAAATAGAGTCTGCAGATTATGAAGAGAACGAAAA
TGACCATACACCCGAAATCATTGCCTCCATGCCGGAGAACTTGAATTTGACACCTCTTTT
GAGCGAGTTCGAATTTCCGAGGTGAACAAGTATTATCTGCTCACGTGTTTGGTGCATTTT
GCAACTAGCGACAAATTTCACTGGCATATTAGTATATCATCAAATCATTAGGATTGCTAT
TTTGTGTCCACTAAGGCGCTCCAGGTGCGTGGAATGGTGCAGTATTAGGCCCTAGCTGTC
CGAGAAAAAAGGAAGCTACACCCTTTGATGTAGTGGGCTCTTTGTGTGTTGTGAGAGGAT
TAAATTGGCCAGATGACACTCTTTCCAAAGATGCAAATAACGAGGAAGAAACATCCAGCC
GGATGCCAGATGTCTGTCTGTGTTGCAAATTCGCTCTTGATCCGGAAAAAGAGTTATTGA
AAAGGGCACTGTCCTGTCTCGTATTGCAGCGTACGAGTGAACCTGAGCGTGTCAATGCAG
GTTCATAAGAAGAAAATTCAGAAGACAGATTGTGACATATTTCTCGCAAGCCATTCACTT
TCCAAGATTCTAGGAGCTTAGAGATCTTGGTGAACAGGGCTCGGGGCAAGTCCATTCCAG
CTTTGTACTTCAACACCATGTCGAATACGGCACCCATGCGTTCGTAGGCTAGCTCGGTGG
TTGCTGGGTCAACATAGCTTTTGATCAGAATGTAAAGAGAGTCTGCTATCTCCAGTAATT
TCATTGATATGTCGGGTCCGTTGCATCTAAAGGCATGATCAGGGAGGTTTTGTAGACTGC
TGAGAAGCTCATTTGCAACTTTTAGAGGAAAGTTAATCTTGAAACATTCGTGTTCGACCG
CAATGCTCTCATCAGAACTAATTAGAAAATTCGCAGACGCAATTAACCAGCCCAAAGACT
GGAGCCAGGCTTTTGAGACAATAACATTCACTTTTTGAGCACTTGTTGCAATTTTATCCA
CATCAATTCTAATTTGGAGCTTCTGTTGGAGCTCAAGGAGCCACAGCCTCTTCAACTCTG
TGGATCCGTTCAAGGTAAAATCCTGAAACAAGTTGGTCTCTGGAATCTGGAACATGTTGT
GAGTGCGCCCTGTCTCGAGCACTTTAGTATGAAGCTCAACATAGAATTGCTTATCCGTTG
CACTGAAAATTGCACACAGAGTGGTGAACCCGTAAAGTGAATTTGAGGATTTTTCGAAAT
CTGCTTCAGGGAGTGGAATTGAAGGTTCAAGAAGAACGGGCAAATTAGTTTTAAAGCTGA
CAAACCTTTCGGTTACCACGAGCAAATAGTAGATTTTTTTCTGCCTATGAGCCTCAACGG
GTGACAAATGAAGCAGTGACTGTGGTTCGTGCATTTTTAAGATATCTGCAAGAGAAACAG
CTTCACGGAGGTAAATAAGACCTTTGTGCTGTTGGCCAGGCGCAATAGCATAGTATTTAT
GTAGGAAGAAGGATGTCAGAAGGAGATCAATATTTGGTGTGCTTTTCAGTCTATACTCAT
AAATTATCCTCCTTGCCTCTTGAGCATATAAGCCAGCATTTTTGAGATCGCTCCTGGCTG
GAGTGTATGTGACGAAACTGTGATATTGTGCGATCGTAGCGCACAATGCGCAACAAAGAG
CATATTCTTGGGCATTGTCCTCGGCGATGGTGATACGAAAATCATCTAAAACATTCGCTA
GTTGAAATACCTGCTCCACATTCTTTAAGGGGCTTGAACGTGTCAAAATTGAAACTGTAC
TTTGAATTGATACTACCGGCCAGATATCGTAAAAATTCGAATGATATATCTCAAGGTAAG
GGATAATTTGTTTCAAAGAAAATGCGCACACATGAGCAGGTAGTGTAGACTGAGCATAGA
TTTCGTCGTTGAGATCCTCTGTGTTTTTGACTGTTCTAGTACTTTTCTTTGGACCGGATT
TATGCCTCACGCGGAGGTTGGTACATGGAACTTTATGAGTGAGACAGTTCGAGCAAATAC
CACCGGTGCATGAATCTATATCGCACTTCACACGGCGGATGGCGCATGCGTCACAGGGTC
TCGGCCGTTGTCGTTTTTTGGTGGACGGCTGCGACATCTCGGTATTTAGATAAGCCAGCT
TGTACTTGTATCCAAAATGTGGGGCTAAAGGGGAGTGTATGATTTTTAATGATATCGGTG
AATGTCCAGAAATTTGGTAAATCGCACACCAAACTTCAACGATGGACTAGCGTGCTAAAC
TGATATTCTACCAAACAACCGTTTGAGTAATTCTGACCATGACAGAGGTCTTGTGTATAT
ATTTTAAGCCCCGTCGATCGACCGCATCGCGACTCCATAAAAAAATTCATTTCACGCGTT
ATTATTATCGTATGAATATGGATCCCCTAAACGTGAAAGTGCAACAGAAACTTAAGGAGC
TCGAAAGCCTTCAGCAGATAAGAGACTTGACGAAACACCTGAATGTTTCACTGGAAGAAT
TTGCTGGCCAAATAGAACTTCTGGGTGAGGAAGCAGGATGCATCGAAACCGTGACGCAAA
ACTGGATGAGAATCATCAGAGCAGTGAGCCTAGCCTCTAACTCTTTGTCGAACTACAAGG
AAGAGGACTATGAAACGGATAGACCAATGACGGAGCGCCTTGTGAGATGCAAGATAGACG
AGAGTCAAAAAATCATCACCAAAAATTGACTTACTTCAATCTATCTAACCATTGTTCTAA
ATTGATCTTGTTGTTCAGTTCCGACAACAGATCGTTCTCCTTCAGCAAATCGTCACGAAG
GCCTTTATCAGCCTCGAGATAATTAATATCGTCCGGATCTAACATTAAGTCCTGAACTTT
AGAGGGAAGTTCTATTTTGAGCGTCGATAACCCGGTCATCATTTTGAAGTATTCATTAGT
ATGCACGTCAGAACCATTCAAAAGCCCATCCCAATCAGTTGTCAAAAACAGCCTTTTCCA
TATCAAGGTTTCCAAGATTTCGTGATAGTGCGAATAACCACTCAGGTTACTTATCACTCT
TGACGGATTGGAGTGTTTGATGACAGTCAACAATTTGAACAAGTTGGCGAAATTGTTGTG
ACTGAATTCATCAAACGTCACTAATGTCAGTAGCTGGACCATCTCAAACAATCCCAGCTG
TTGCTGCTTATCCATCTTTGCCACAATATCGCCGATCTGACTTTCAAAATGTGTAAACTT
GTTATCAACCAAGTATCTTGAGTCTTGTATGAATCCATCTTTAGTTTTTGAATCAATTAA
TCTCGCATAATTCAATTGAAGCTCAATCATTTGCAAGTGTATTTCTATCTCATCCAACAG
ATTGTCGTGATCCTCATTCAAAGCTATCAGTGAAAGTTTGGCAATATTCAGTTCGAACTT
TTTCTTTTCAATTTCCTCCGACTCTGCAGTGGAAACATATGCAATCAGCTTTTCTGCTGC
CTTGAAATACTCATCGTTTTCCACGTCAAGTATCCAGGCAAATTTATAGTTGTTGATGCT
TGAATTCAAAAACATGCGCACGTAATCTGGATATTGAGGGATGTAATTGAGGATTAGCCG
AATCTTTCTGGTTCTAGCATAGAATCCAAAAAGCACTTCTGCAAAACGGTATCCATACCG
CTGGAAACATTCCTAGTTTATGTTGTATCTATG
(SEQ ID NO: 5) (fragment amplified with KP-MAL7 and KP-MAL8)
CATACCGCTGGAAACATTCCTAGTTTATGTTGTATCTATGAATATTTTTTTACATGGTGG
ATTTGTTCCAATCTAGTTGTTAGTTATTTGTCGTTATAGTCACTAATACAGTTACCATCG
GAATCTTTTCATTTCTTTTTATACGTACGTATATGTACTAGATGAAGAATGCGACAAGGC
CGACCAACAGCAATGGTGCTTGGTACCAAAGTTTGGAAGGTGCTACCGAATTGGCCGATG
ATATTGAAGTGGAGTTGTCATTCTCCTCATTGGTTGCTCCAGTAGTGGTAAAATTAACGG
GGATATCTTCGGTGGAAGTGTCAATATCACCGTTCCAACAATAATTCTCAAAACATCTTC
TACATTGCTCTGTTTGTGTTGCATTGGATCTCTCCTGCTGTCTACGAATGATTGCACATC
CAACACAAGTACGCCATTCTTCATCAATAGTCATGTTCAAACGAGACGACACTTCAAAAC
CGTTTTGGATCATTCCTCTCTTCTCGGATTCAGAGTAGTCTAACTTGAAAGTTGAAGTGT
TACTCCAATACGAGAAAGGTCTATTGGCCAGATAAACAACCAGGGGAGGAATGTGATCCG
TGCCTTCAACAATGTCTGTCAAATTTCTAGCATCACAACCAAAGAAGGTTGGCTTACTCG
AAAGATTCAAGTTTAGGAAAGTGGTGGTATCAGGTACATAAGGGAACGTTGTTCCATTTG
CTTGAGAAGAAAACTGTCTCATGTAGGTGTTGACTAATGATGAACCGTTTGGCCAAGAGA
GGTCAGTGTCTGCACTGTTGTCAAACGCAAAGATGATATCAACGTCACGCTCCTTTTGAA
GTAGAGGCTGCAGAGGGACGTTTTGGTTATCCTCTCCACCATCAACCAAGTAAAGGGTGT
CATTTTCCGCAATGGCACCTACTCCTGCATACGTACTTTTGTAGAAAGGGTTGGGGGAAT
AGATAGCAATGTCGTCTTCGTCTTCGCTAAGTCCAGTCAG
(SEQ ID NO: 15):
Sequence of plasmid vector into which MAL promoter has been
cloned
AGATCTGCTTGCCCATTCTTAATCCGGCTGAATTTTGGTGCGTGATCGCGGCCCAAGTTT
GCTTCCAGAAAGAGCTTAGAATAATGCTTTTTACCTTTGATGAATGCTCGCGAGTGCTCG
TTTTAATTACCCCATACGCATTGTTGCAGTATGCAAGCACCATTGCAAATCACGCATGGA
AGAATTCTGCAAACACGTATAGCCCCGCGTTGTTGAGGAGGAGGACTTAGTGTAAGACCA
TAAAGCTGTGTATTATGGTGAGCTCTGTTGTCTGGTGACCCTCTGTTTTGGCCACGCCTT
GGAAAACACACTCTGGCAAATCCTCATTTTTGTGCATGAGATTCGAGCCAACAGCACAAT
AGGACGCTCGCGGCCTATTGCTCCACGAAATAATTGCGAAATCTAAATTATGACGCAAGG
CTGAGAGATTCTGGCACGCCGCATTTGCGGGGAAATAATAATCGGGGAATTTTCCGGGGT
TCGGGATGGGGCTTGGAAAGAAGGTTCAACACAGACCAAAACAACTTGGCAATGCACGCA
GACGTTTATGCGATACCCCGAGCCAGCTTAGGACGAGTTAGATGAAGAAGAGCCCCGCAA
AAAGAGTTTCGGCGCGTTGGACAAACATTGATACTCCACGGCAAATTAACTACAGTTCCG
TGTATGCGGATAGGGATATCTTCGGGAGTATCGCAATAGAAAGCAGGCACTGTGCAGATT
ACGCGACATGATAGCTTTGTATGTTCTACAGACTCTGCCGTACCACTCTACATATAATGT
GAGAGCTCTGCAGCGTCGTAATGAACACTTGGGTTATACAAGTTTCCTAAGAGCCCTTTG
ACGTTGATTGCTCTGGCTTCCCTACGGGCTCTCATGTGGTTCAAGTGACTCCGCAGTGGC
TAGCAAGCGTGGGGGTCAATTACGTCACTTCTATTCATGTACCCCAGACTCAATTGTTGG
CAGTTATTTCAGCGAGAATTAAATGTGCAAATTAATTCTCGCTCAAATAAATTAGGCCGT
GATTTAATACTCGCTGAAACAGGATCTTGTCTGGGGTACAGATAACAATCAAATAACTAT
TATGGACGTGCATAGGAGGTGGAGCCCATGACGCAAACGGAAATCCTCTTTTTATTTTCG
GGAAGTTAGGAGGATACCAAAGCGTCGCGCTCGCTAAAGTGATGAGAATGTCTTTAGTAA
GCTCAAGCCATATAAAGACCCTCCGCCTCCACAATTTTTTTATCCCTCTTGACAATATTA
ATTCCTTATGAGATTTCCTTCAATTTTTACTGCTGTTTTATTCGCAGCATCCTCCGCATT
AGCTGCTCCAGTCAACACTACAACAGAAGATGAAACGGCACAAATTCCGGCTGAAGCTGT
CATCGGTTACTCAGATTTAGAAGGGGATTTCGATGTTGCTGTTTTGCCATTTTCCAACAG
CACAAATAACGGGTTATTGTTTATAAATACTACTATTGCCAGCATTGCTGCTAAAGAAGA
AGGGGTATCTCTCGAGAAAAGAGAGGCTGAAGCTGGAGCACCAGGTGCCCCAGGCTTGCA
AGGAGCTCCTGGCCTGCAGGGTATGCCAGGAGAGAGAGGTGCCGCTGGCTTGCCAGGTCC
AAAGGGAGAGAGAGGTGATGCCGGTCCAAAGGGCGCTGATGGAGCACCGGGTGCTCCAGG
TTTGCAGGGAATGCCTGGCGAAAGAGGCGCCGCCGGATTGCCAGGTCCAAAAGGCGAACG
TGGAGATGCTGGTCCTAAGGGTGCAGATGGAGCTCCAGGTAAAGACGGCGTTAGAGGTCT
GGCAGGTCCAATTGGACCTCCAGGTGAGAGAGGAGCTGCAGGATTGCCAGGCCCAAAGGG
TGAACGTGGCGACGCCGGTCCAAAGGGAGCAGATGGAGCTCCGGGTAAAGATGGAGTGAG
AGGACTGGCAGGCCCTATCGGTCCACCAGGACCAGCTGGTGCCCCAGGCGCTCCTGGTTT
GCAAGGTATGCCTGGAGAGAGAGGTGCAGCTGGACTGCCAGGCCCAAAAGGTGAAAGAGG
TGATGCCGGACCAAAAGGCGCCGATGGTGCTCCAGGTAAAGATGGCGTTAGAGGTCTTGC
TGGTCCACCCGGAGCCCCTGGCTTGCAGGGCGCACCAGGATTGCAGGGTATGCCAGGCGA
GAGAGGAGCTGCAGGTCTGCCTGGTCCAAAAGGTGAAAGAGGAGATGCTGGTCCTAAGGG
TGCTGACGGCGCCCCTGGAGCTCCAGGATTGCAGGGAATGCCAGGTGAGAGAGGCGCTGC
CGGTTTGCCAGGACCTAAGGGTGAAAGAGGTGATGCTGGCCCAAAAGGTGCCGATGGAGC
TCCAGGAAAAGATGGTGTTAGAGGACTGGCCGGTCCTATCGGCCCTCCTGGAGAAAGAGG
AGCTGCTGGTTTGCCTGGCCCAAAAGGCGAGAGAGGTGACGCTGGCCCAAAAGGAGCCGA
CGGTGCTCCTGGCAAGGACGGAGTGAGAGGCCTGGCCGGTCCTATTGGTCCACCAGGACC
TGCTGGCGCTCCAGGAGCTCCTGGTTTGCAGGGTATGCCAGGTGAAAGAGGAGCCGCAGG
CCTGCCAGGTCCTAAGGGTGAGAGAGGTGACGCCGGTCCTAAAGGCGCTGATGGAGCTCC
TGGTAAAGATGGCGTTAGAGGTTTGGCCGGACCCCCAGGTGCCCCAGGTCTGCAAGGAGC
TCCAGGTCTTCAGGGTATGCCAGGCGAAAGAGGAGCTGCTGGTTTGCCAGGCCCAAAGGG
TGAAAGAGGAGATGCCGGCCCAAAAGGAGCTGACGGTGCACCAGGCGCTCCAGGCCTGCA
GGGTATGCCAGGAGAAAGAGGTGCTGCTGGTTTGCCAGGACCAAAGGGAGAAAGAGGCGA
CGCCGGTCCAAAGGGTGCAGATGGAGCTCCTGGCAAAGACGGAGTGAGAGGCTTGGCAGG
TCCAATCGGACCGCCAGGCGAGAGAGGTGCTGCCGGCTTGCCAGGTCCAAAGGGTGAAAG
AGGAGACGCAGGTCCTAAAGGTGCTGACGGCGCACCAGGAAAAGATGGTGTTAGAGGTCT
GGCTGGACCTATCGGCCCACCAGGACCAGCTGGTGCTCCAGGTGCTCCTGGCCTTCAGGG
CATGCCAGGAGAAAGAGGTGCTGCCGGTCTGCCTGGACCTAAGGGCGAAAGAGGCGATGC
AGGTCCAAAGGGTGCTGATGGAGCTCCTGGTAAAGATGGTGTTAGAGGATTGGCAGGCCC
ACCCGGGTAATAGTCTAGAGTCGACTGAGTTTGTAGCCTTAGACATGACTGTTCCTCAGT
TCAAGTTGGGCACTTACGAGAAGACCGGTCTTGCTAGATTCTAATCAAGAGGATGTCAGA
ATGCCATTTGCCTGAGAGATGCAGGCTTCATTTTTGATACTTTTTTATTTGTAACCTATA
TAGTATAGGATTTTTTTTGTCATTTTGTTTCTTCTCGTACGAGCTTGCTCCTGATCAGCC
TATCTCGCAGCTGATGAATATCTTGTGGTAGGGGTTTGGGAAAATCATTCGAGTTTGATG
TTTTTCTTGGTATTTCCCACTCCTCTTCAGAGTACAGAAGATTAAGTGAGACCTTCGTTT
GTGCACTAGTCCCACACACCATAGCTTCAAAATGTTTCTACTCCTTTTTTACTCTTCCAG
ATTTTCTCGGACTCCGCGCATCGCCGTACCACTTCAAAACACCCAAGCACAGCATACTAA
ATTTTCCCTCTTTCTTCCTCTAGGGTGTCGTTAATTACCCGTACTAAAGGTTTGGAAAAG
AAAAAAGAGACCGCCTCGTTTCTTTTTCTTCGTCGAAAAAGGCAATAAAAATTTTTATCA
CGTTTCTTTTTCTTGAAATTTTTTTTTTTAGTTTTTTTCTCTTTCAGTGACCTCCATTGA
TATTTAAGTTAATAAACGGTCTTCAATTTCTCAAGTTTCAGTTTCATTTTTCTTGTTCTA
TTACAACTTTTTTTACTTCTTGTTCATTAGAAAGAAAGCATAGCAATCTAATCTAAGGGG
CGGTGTTGACAATTAATCATCGGCATAGTATATCGGCATAGTATAATACGACAAGGTGAG
GAACTAAACCATGGCCAAGTTGACCAGTGCCGTTCCGGTGCTCACCGCGCGCGACGTCGC
CGGAGCGGTCGAGTTCTGGACCGACCGGCTCGGGTTCTCCCGCGACTTCGTGGAGGACGA
CTTCGCCGGTGTGGTCCGGGACGACGTGACCCTGTTCATCAGCGCGGTCCAGGACCAGGT
GGTGCCGGACAACACCCTGGCCTGGGTGTGGGTGCGCGGCCTGGACGAGCTGTACGCCGA
GTGGTCGGAGGTCGTGTCCACGAACTTCCGGGACGCCTCCGGGCCGGCCATGACCGAGAT
CGGCGAGCAGCCGTGGGGGCGGGAGTTCGCCCTGCGCGACCCGGCCGGCAACTGCGTGCA
CTTCGTCGCCGAGGAGCAGGACTGACACGTCCGACGGCGGCCCACGGGTCCCAGGCCTCG
GAGATCCGTCCCCCTTTTCCTTTGTCGATATCATGTAATTAGTTATGTCACGCTTACATT
CACGCCCTCCCCCCACATCCGCTCTAACCGAAAAGGAAGGAGTTAGACAACCTGAAGTCT
AGGTCCCTATTTATTTTTTTATAGTTATGTTAGTATTAAGAACGTTATTTATATTTCAAA
TTTTTCTTTTTTTTCTGTACAGACGCGTGTACGCATGTAACATTATACTGAAAACCTTGC
TTGAGAAGGTTTTGGGACGCTCGAAGGCTTTAATTTGCAAGCTGGAGACCAACATGTGAG
CAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTG
TTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGAT
C
(SEQ ID NO: 16):
P-mal: sucrose-inducible MAL promoter (included in MAL cluster)
GCTTGCCCATTCTTAATCCGGCTGAATTTTGGTGCGTGATCGCGGCCCAAGTTTGCTTCC
AGAAAGAGCTTAGAATAATGCTTTTTACCTTTGATGAATGCTCGCGAGTGCTCGTTTTAA
TTACCCCATACGCATTGTTGCAGTATGCAAGCACCATTGCAAATCACGCATGGAAGAATT
CTGCAAACACGTATAGCCCCGCGTTGTTGAGGAGGAGGACTTAGTGTAAGACCATAAAGC
TGTGTATTATGGTGAGCTCTGTTGTCTGGTGACCCTCTGTTTTGGCCACGCCTTGGAAAA
CACACTCTGGCAAATCCTCATTTTTGTGCATGAGATTCGAGCCAACAGCACAATAGGACG
CTCGCGGCCTATTGCTCCACGAAATAATTGCGAAATCTAAATTATGACGCAAGGCTGAGA
GATTCTGGCACGCCGCATTTGCGGGGAAATAATAATCGGGGAATTTTCCGGGGTTCGGGA
TGGGGCTTGGAAAGAAGGTTCAACACAGACCAAAACAACTTGGCAATGCACGCAGACGTT
TATGCGATACCCCGAGCCAGCTTAGGACGAGTTAGATGAAGAAGAGCCCCGCAAAAAGAG
TTTCGGCGCGTTGGACAAACATTGATACTCCACGGCAAATTAACTACAGTTCCGTGTATG
CGGATAGGGATATCTTCGGGAGTATCGCAATAGAAAGCAGGCACTGTGCAGATTACGCGA
CATGATAGCTTTGTATGTTCTACAGACTCTGCCGTACCACTCTACATATAATGTGAGAGC
TCTGCAGCGTCGTAATGAACACTTGGGTTATACAAGTTTCCTAAGAGCCCTTTGACGTTG
ATTGCTCTGGCTTCCCTACGGGCTCTCATGTGGTTCAAGTGACTCCGCAGTGGCTAGCAA
GCGTGGGGGTCAATTACGTCACTTCTATTCATGTACCCCAGACTCAATTGTTGGCAGTTA
TTTCAGCGAGAATTAAATGTGCAAATTAATTCTCGCTCAAATAAATTAGGCCGTGATTTA
ATACTCGCTGAAACAGGATCTTGTCTGGGGTACAGATAACAATCAAATAACTATTATGGA
CGTGCATAGGAGGTGGAGCCCATGACGCAAACGGAAATCCTCTTTTTATTTTCGGGAAGT
TAGGAGGATACCAAAGCGTCGCGCTCGCTAAAGTGATGAGAATGTCTTTAGTAAGCTCAA
GCCATATAAAGACCCTCCGCCTCCACAATTTTTTTATCCCTCTTGACAATATTAATTCCT
T
(SEQ ID NO: 17):
MF-a: signal sequence of target protein
ATGAGATTTCCTTCAATTTTTACTGCTGTTTTATTCGCAGCATCCTCCGCATTAGCTGCT
CCAGTCAACACTACAACAGAAGATGAAACGGCACAAATTCCGGCTGAAGCTGTCATCGGT
TACTCAGATTTAGAAGGGGATTTCGATGTTGCTGTTTTGCCATTTTCCAACAGCACAAAT
AACGGGTTATTGTTTATAAATACTACTATTGCCAGCATTGCTGCTAAAGAAGAAGGGGTA
TCTCTCGAGAAAAGAGAGGCTGAAGCT
(SEQ ID NO: 18):
NRC3: target protein (CBE3)
GGAGCACCAGGTGCCCCAGGCTTGCAAGGAGCTCCTGGCCTGCAGGGTATGCCAGGAGAG
AGAGGTGCCGCTGGCTTGCCAGGTCCAAAGGGAGAGAGAGGTGATGCCGGTCCAAAGGGC
GCTGATGGAGCACCGGGTGCTCCAGGTTTGCAGGGAATGCCTGGCGAAAGAGGCGCCGCC
GGATTGCCAGGTCCAAAAGGCGAACGTGGAGATGCTGGTCCTAAGGGTGCAGATGGAGCT
CCAGGTAAAGACGGCGTTAGAGGTCTGGCAGGTCCAATTGGACCTCCAGGTGAGAGAGGA
GCTGCAGGATTGCCAGGCCCAAAGGGTGAACGTGGCGACGCCGGTCCAAAGGGAGCAGAT
GGAGCTCCGGGTAAAGATGGAGTGAGAGGACTGGCAGGCCCTATCGGTCCACCAGGACCA
GCTGGTGCCCCAGGCGCTCCTGGTTTGCAAGGTATGCCTGGAGAGAGAGGTGCAGCTGGA
CTGCCAGGCCCAAAAGGTGAAAGAGGTGATGCCGGACCAAAAGGCGCCGATGGTGCTCCA
GGTAAAGATGGCGTTAGAGGTCTTGCTGGTCCACCCGGAGCCCCTGGCTTGCAGGGCGCA
CCAGGATTGCAGGGTATGCCAGGCGAGAGAGGAGCTGCAGGTCTGCCTGGTCCAAAAGGT
GAAAGAGGAGATGCTGGTCCTAAGGGTGCTGACGGCGCCCCTGGAGCTCCAGGATTGCAG
GGAATGCCAGGTGAGAGAGGCGCTGCCGGTTTGCCAGGACCTAAGGGTGAAAGAGGTGAT
GCTGGCCCAAAAGGTGCCGATGGAGCTCCAGGAAAAGATGGTGTTAGAGGACTGGCCGGT
CCTATCGGCCCTCCTGGAGAAAGAGGAGCTGCTGGTTTGCCTGGCCCAAAAGGCGAGAGA
GGTGACGCTGGCCCAAAAGGAGCCGACGGTGCTCCTGGCAAGGACGGAGTGAGAGGCCTG
GCCGGTCCTATTGGTCCACCAGGACCTGCTGGCGCTCCAGGAGCTCCTGGTTTGCAGGGT
ATGCCAGGTGAAAGAGGAGCCGCAGGCCTGCCAGGTCCTAAGGGTGAGAGAGGTGACGCC
GGTCCTAAAGGCGCTGATGGAGCTCCTGGTAAAGATGGCGTTAGAGGTTTGGCCGGACCC
CCAGGTGCCCCAGGTCTGCAAGGAGCTCCAGGTCTTCAGGGTATGCCAGGCGAAAGAGGA
GCTGCTGGTTTGCCAGGCCCAAAGGGTGAAAGAGGAGATGCCGGCCCAAAAGGAGCTGAC
GGTGCACCAGGCGCTCCAGGCCTGCAGGGTATGCCAGGAGAAAGAGGTGCTGCTGGTTTG
CCAGGACCAAAGGGAGAAAGAGGCGACGCCGGTCCAAAGGGTGCAGATGGAGCTCCTGGC
AAAGACGGAGTGAGAGGCTTGGCAGGTCCAATCGGACCGCCAGGCGAGAGAGGTGCTGCC
GGCTTGCCAGGTCCAAAGGGTGAAAGAGGAGACGCAGGTCCTAAAGGTGCTGACGGCGCA
CCAGGAAAAGATGGTGTTAGAGGTCTGGCTGGACCTATCGGCCCACCAGGACCAGCTGGT
GCTCCAGGTGCTCCTGGCCTTCAGGGCATGCCAGGAGAAAGAGGTGCTGCCGGTCTGCCT
GGACCTAAGGGCGAAAGAGGCGATGCAGGTCCAAAGGGTGCTGATGGAGCTCCTGGTAAA
GATGGTGTTAGAGGATTGGCAGGCCCACCCGGG
(SEQ ID NO: 19):
pPICZa-CBE3
AGATCTAACATCCAAAGACGAAAGGTTGAATGAAACCTTTTTGCCATCCGACATCCACAG
GTCCATTCTCACACATAAGTGCCAAACGCAACAGGAGGGGATACACTAGCAGCAGACCGT
TGCAAACGCAGGACCTCCACTCCTCTTCTCCTCAACACCCACTTTTGCCATCGAAAAACC
AGCCCAGTTATTGGGCTTGATTGGAGCTCGCTCATTCCAATTCCTTCTATTAGGCTACTA
ACACCATGACTTTATTAGCCTGTCTATCCTGGCCCCCCTGGCGAGGTTCATGTTTGTTTA
TTTCCGAATGCAACAAGCTCCGCATTACACCCGAACATCACTCCAGATGAGGGCTTTCTG
AGTGTGGGGTCAAATAGTTTCATGTTCCCCAAATGGCCCAAAACTGACAGTTTAAACGCT
GTCTTGGAACCTAATATGACAAAAGCGTGATCTCATCCAAGATGAACTAAGTTTGGTTCG
TTGAAATGCTAACGGCCAGTTGGTCAAAAAGAAACTTCCAAAAGTCGGCATACCGTTTGT
CTTGTTTGGTATTGATTGACGAATGCTCAAAAATAATCTCATTAATGCTTAGCGCAGTCT
CTCTATCGCTTCTGAACCCCGGTGCACCTGTGCCGAAACGCAAATGGGGAAACACCCGCT
TTTTGGATGATTATGCATTGTCTCCACATTGTATGCTTCCAAGATTCTGGTGGGAATACT
GCTGATAGCCTAACGTTCATGATCAAAATTTAACTGTTCTAACCCCTACTTGACAGCAAT
ATATAAACAGAAGGAAGCTGCCCTGTCTTAAACCTTTTTTTTTATCATCATTATTAGCTT
ACTTTCATAATTGCGACTGGTTCCAATTGACAAGCTTTTGATTTTAACGACTTTTAACGA
CAACTTGAGAAGATCAAAAAACAACTAATTATTCGAAGAATCCAAACGATGAGATTTCCT
TCAATTTTTACTGCAGTTTTATTCGCAGCATCCTCCGCATTAGCTGCTCCAGTCAACACT
ACAACAGAAGATGAAACGGCACAAATTCCGGCTGAAGCTGTCATCGGTTACTCAGATTTA
GAAGGGGATTTCGATGTTGCTGTTTTGCCATTTTCCAACAGCACAAATAACGGGTTATTG
TTTATAAATACTACTATTGCCAGCATTGCTGCTAAAGAAGAAGGGGTATCTCTCGAGAAA
AGAGAGGCTGAAGCTGGAGCTCCAGGTGCTCCAGGCCTTCAGGGTGCTCCAGGTTTGCAG
GGTATGCCAGGAGAGAGAGGAGCTGCCGGTTTGCCTGGCCCAAAGGGTGAGAGAGGTGAC
GCTGGTCCTAAAGGTGCTGACGGAGCTCCAGGAGCTCCTGGACTGCAAGGCATGCCTGGT
GAAAGAGGTGCAGCTGGCCTGCCAGGACCAAAAGGAGAAAGAGGCGATGCAGGCCCAAAG
GGAGCAGATGGTGCCCCAGGTAAAGACGGTGTTAGAGGCTTGGCTGGACCTATCGGACCT
CCAGGTGAAAGAGGTGCAGCTGGCCTGCCAGGACCAAAAGGAGAAAGAGGCGATGCAGGC
CCAAAGGGAGCAGATGGTGCCCCAGGTAAAGACGGTGTTAGAGGCTTGGCTGGACCTATC
GGACCACCTGGTCCAGCTGGAGCCCCTGGTGCTCCAGGTTTGCAGGGTATGCCAGGAGAG
AGAGGAGCTGCCGGTTTGCCTGGTCCAAAGGGCGAAAGAGGTGATGCCGGACCGAAAGGC
GCTGATGGCGCCCCAGGCAAGGATGGCGTGAGAGGTCTGGCCGGTCCACCCGGTGCTCCA
GGCCTTCAGGGTGCTCCAGGTTTGCAGGGTATGCCAGGAGAGAGAGGAGCTGCCGGTTTG
CCTGGCCCAAAGGGTGAGAGAGGTGACGCTGGTCCTAAAGGTGCTGACGGAGCTCCAGGA
GCTCCTGGACTGCAAGGCATGCCTGGTGAAAGAGGTGCAGCTGGCCTGCCAGGACCAAAA
GGAGAAAGAGGCGATGCAGGCCCAAAGGGAGCAGATGGTGCCCCAGGTAAAGACGGTGTT
AGAGGCTTGGCTGGACCTATCGGACCTCCAGGTGAAAGAGGTGCAGCTGGCCTGCCAGGA
CCAAAAGGAGAAAGAGGCGATGCAGGCCCAAAGGGAGCAGATGGTGCCCCAGGTAAAGAC
GGTGTTAGAGGCTTGGCTGGACCTATCGGACCACCTGGTCCAGCTGGAGCCCCTGGTGCT
CCAGGTTTGCAGGGTATGCCAGGAGAGAGAGGAGCTGCCGGTTTGCCTGGTCCAAAGGGC
GAAAGAGGTGATGCCGGACCGAAAGGCGCTGATGGCGCCCCAGGCAAGGATGGCGTGAGA
GGTCTGGCCGGTCCACCCGGTGCTCCAGGCCTTCAGGGTGCTCCAGGTTTGCAGGGTATG
CCAGGAGAGAGAGGAGCTGCCGGTTTGCCTGGCCCAAAGGGTGAGAGAGGTGACGCTGGT
CCTAAAGGTGCTGACGGAGCTCCAGGAGCTCCTGGACTGCAAGGCATGCCTGGTGAAAGA
GGTGCAGCTGGCCTGCCAGGACCAAAAGGAGAAAGAGGCGATGCAGGCCCAAAGGGAGCA
GATGGTGCCCCAGGTAAAGACGGTGTTAGAGGCTTGGCTGGACCTATCGGACCTCCAGGT
GAAAGAGGTGCAGCTGGCCTGCCAGGACCAAAAGGAGAAAGAGGCGATGCAGGCCCAAAG
GGAGCAGATGGTGCCCCAGGTAAAGACGGTGTTAGAGGCTTGGCTGGACCTATCGGACCA
CCTGGTCCAGCTGGAGCCCCTGGTGCTCCAGGTTTGCAGGGTATGCCAGGAGAGAGAGGA
GCTGCCGGTTTGCCTGGTCCAAAGGGCGAAAGAGGTGATGCCGGACCGAAAGGCGCTGAT
GGCGCCCCAGGCAAGGATGGCGTGAGAGGTCTGGCCGGTCCACCCGGGTAAGCGGCCGCC
AGCTTTCTAGAACAAAAACTCATCTCAGAAGAGGATCTGAATAGCGCCGTCGACCATCAT
CATCATCATCATTGAGTTTGTAGCCTTAGACATGACTGTTCCTCAGTTCAAGTTGGGCAC
TTACGAGAAGACCGGTCTTGCTAGATTCTAATCAAGAGGATGTCAGAATGCCATTTGCCT
GAGAGATGCAGGCTTCATTTTTGATACTTTTTTATTTGTAACCTATATAGTATAGGATTT
TTTTTGTCATTTTGTTTCTTCTCGTACGAGCTTGCTCCTGATCAGCCTATCTCGCAGCTG
ATGAATATCTTGTGGTAGGGGTTTGGGAAAATCATTCGAGTTTGATGTTTTTCTTGGTAT
TTCCCACTCCTCTTCAGAGTACAGAAGATTAAGTGAGACCTTCGTTTGTGCGGATCCCCC
ACACACCATAGCTTCAAAATGTTTCTACTCCTTTTTTACTCTTCCAGATTTTCTCGGACT
CCGCGCATCGCCGTACCACTTCAAAACACCCAAGCACAGCATACTAAATTTCCCCTCTTT
CTTCCTCTAGGGTGTCGTTAATTACCCGTACTAAAGGTTTGGAAAAGAAAAAAGAGACCG
CCTCGTTTCTTTTTCTTCGTCGAAAAAGGCAATAAAAATTTTTATCACGTTTCTTTTTCT
TGAAAATTTTTTTTTTTGATTTTTTTCTCTTTCGATGACCTCCCATTGATATTTAAGTTA
ATAAACGGTCTTCAATTTCTCAAGTTTCAGTTTCATTTTTCTTGTTCTATTACAACTTTT
TTTACTTCTTGCTCATTAGAAAGAAAGCATAGCAATCTAATCTAAGGGCGGTGTTGACAA
TTAATCATCGGCATAGTATATCGGCATAGTATAATACGACAAGGTGAGGAACTAAACCAT
GGCCAAGTTGACCAGTGCCGTTCCGGTGCTCACCGCGCGCGACGTCGCCGGAGCGGTCGA
GTTCTGGACCGACCGGCTCGGGTTCTCCCGGGACTTCGTGGAGGACGACTTCGCCGGTGT
GGTCCGGGACGACGTGACCCTGTTCATCAGCGCGGTCCAGGACCAGGTGGTGCCGGACAA
CACCCTGGCCTGGGTGTGGGTGCGCGGCCTGGACGAGCTGTACGCCGAGTGGTCGGAGGT
CGTGTCCACGAACTTCCGGGACGCCTCCGGGCCGGCCATGACCGAGATCGGCGAGCAGCC
GTGGGGGCGGGAGTTCGCCCTGCGCGACCCGGCCGGCAACTGCGTGCACTTCGTGGCCGA
GGAGCAGGACTGACACGTCCGACGCGGCCCGACGGGTCCGAGGCCTCGGAGATCCGTCCC
CCTTTTCCTTTGTCGATATCATGTAATTAGTTATGTCACGCTTACATTCACGCCCTCCCC
CCACATCCGCTCTAACCGAAAAGGAAGGAGTTAGACAACCTGAAGTCTAGGTCCCTATTT
ATTTTTTTATAGTTATGTTAGTATTAAGAACGTTATTTATATTTCAAATTTTTCTTTTTT
TTCTGTACAGACGCGTGTACGCATGTAACATTATACTGAAAACCTTGCTTGAGAAGGTTT
TGGGACGCTCGAAGGCTTTAATTTGCAAGCTGGAGACCAACATGTGAGCAAAAGGCCAGC
AAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGA
GGTATGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATC

[Sequence list] International application 23F00372WIJP24020485_2.xml based on International Patent Cooperation Treaty

What is claimed is:

1. A recombinant yeast comprising:
a first exogenous sucrose-inducible promoter for expressing a target protein.

2. The recombinant yeast according to claim 1, further comprising:
an exogenous gene encoding the target protein.

3. The recombinant yeast according to claim 1, further comprising:
an exogenous gene that confers sucrose assimilation.

4. The recombinant yeast according to claim 3,
wherein the exogenous gene that confers sucrose assimilation is linked to a second exogenous sucrose-inducible promoter.

5. The recombinant yeast according to claim 3,
wherein the exogenous gene that confers sucrose assimilation is a maltase gene.

6. The recombinant yeast according to claim 3,
wherein the first exogenous sucrose-inducible promoter and the exogenous gene that confers sucrose assimilation are derived from a yeast having an α-glucosidase which has sucrose-hydrolyzing activity.

7. The recombinant yeast according to claim 6,
wherein the yeast having an α-glucosidase which has sucrose-hydrolyzing activity belongs to any of the genus Ogataea, Candida, Cyberlindnera, Wickerhamomyces, Clavispora, Debaryomyces, Meyerozyma, Scheffersomyces, Metschnikowia, Spathaspora, Babjeviella, Hypopichia, or Yamadazyma.

8. The recombinant yeast according to claim 7,
wherein the yeast having an α-glucosidase which has sucrose-hydrolyzing activity is Ogataea parapolymorpha.

9. The recombinant yeast according to claim 1,
wherein the recombinant yeast is the genus Komagataella.

10. A method for producing a target protein, comprising:
culturing the recombinant yeast according to claim 1 in a culture medium containing sucrose as a carbon source.

11. The method for producing a target protein according to claim 10,
wherein the target protein is an antibody, a bioactive protein, a bioactive polypeptide, an extracellular matrix, an artificial protein, or an enzyme.

12. The method for producing a target protein according to claim 10,
wherein a concentration of the sucrose in the culture medium during the culture is 0.5 to 2 g/mL.

13. The method for producing a target protein according to claim 10,
wherein an addition rate of the sucrose is 2 to 100 g/hour per 0.9 L of the culture medium at the start of the culture.

14. A kit for producing the recombinant yeast according to claim 1, the kit comprising:
a yeast; and
an expression construct containing a sucrose-inducible promoter.

15. The kit according to claim 14,
wherein the expression construct contains an exogenous gene encoding the target protein, and
the sucrose-inducible promoter induces expression of the exogenous gene encoding the target protein.

16. A method for producing a recombinant yeast, which is a method for producing the recombinant yeast according to claim 1, the method comprising:
introducing an expression construct containing a sucrose-inducible promoter into a yeast.

17. The method for producing a recombinant yeast according to claim 16,
wherein the expression construct containing the sucrose-inducible promoter is introduced into a genome of the yeast.

18. The method for producing a recombinant yeast according to claim 16,
wherein the expression construct containing the sucrose-inducible promoter contains an exogenous gene encoding the sucrose-inducible promoter and the target protein, and
the sucrose-inducible promoter is an expression construct that induces expression of an exogenous gene encoding the target protein.

19. The method for producing a recombinant yeast according to claim 16,
wherein the expression construct containing the sucrose-inducible promoter is introduced into a genome of a yeast having an exogenous gene that confers sucrose assimilation.


SEQUENCE LISTING (19 sequences)

SEQ ID NO: 1 - DNA, 10511 bp, Ogataea parapolymorpha
  source 1..10511: mol_type = genomic DNA
gtattcccggaaagaactcgggatacggccggattattagctaaacgagccatgttagcaaaacggttggcgaggtttatttttttatttacatttcactgcaagtcgatcgaccgtgtcgcgactaatgaataatgaatatattatgaaatgtcaagattcaactagtctatcccaatctggctccgcaaacgcttcgactgtctcataaaacaaagaaaatattcaactatatacctttattaggctgtaaaatctaattgacctcgatcagtctaccctcatatggttgcagcttgcctgtgatgttgtttgcaatattagacaggagcagctttccttgagggattgggtactcgacttctctatcagtaaagttcaaaacaacagccatcttcttttggcctttgttgctggtcttggtgtagtagaaaacctcctggttatcgtagtcttgcacctcgaaacggccaaaagtcaacgtctcaggataaagctttctggtcttgatggcatttctgtaaaagttgagcacagagttagggtcatctttctgcgaagcaacgttgatgtctttgtagttgtcgttgattctcatccaaggcttaccaccgaaaccaccgttttcagaagagtcccactgaactggagtccttgcattgtctcttgccagcaaattgatgatcttcagcagctcggccttctcctcatcagagtgctcggtctcgttgaacgcgttccagtagttaatggtgttgatatccaaatactctgaaatatcccatttcggtgaaacatttgtcataccaatctcttgaccttggtacacaaacagagtgccggtcaaggttgtctgcaacagtgccagcatctttgcagatctgctccaaaacagtttgttggaggtgtttccgaaccttgtgacacagcgtggttggtcgtggttctcgataaaaacggtggaccatgcgtcattcagctcaccggtctcgtcatcaaagatgaattctgactggttgatgatggcatccttgaagtcggtcaagttgaagcctttgtatctaaaacggtcgctcttgtcggatccaacgtcaacagtgtcgaacaggaacatcatgttcatctccttttcttttgcagagacgtacttgagagcatctgccttggagcagtgaccaacttctccgacagtcatggcatcatacctggaagtcactttctcgtacatctctttgtggaactcgtgaatacgtggaccggagttgatgagtggaccagccggctggtatttctcacctgggaaggtgattggcgcatcctcaaaagtctggacttttgagtagaggccagcagtgtcgattctgaaaccagaaacgcccttctcgtaccaaaactcaagagcagatttgtaaatggcttcacgggtctctgggttctcccagttcaaatcaggctgagtctcagcaaacaggcgaaggtagtattgcttagtcttttcgtcataagcccatgcagaaccagagaaaaaggaaagccagttgttggcattgtccttccaaatgtaccaatctctctttggattgtctctgctagatctagactccttgaaccaagcgtgctcagacgaggtgtggttaataacgaggtccagaatcaatctcatgtctctcttcttgatctcggcaagcagtctgtccatgtcctccatagtgccaaattgagggttgatggcattgtagtcagaaatatcgtatcccatatcgtccaatggcgaagcatagtgcggagaaagccagatcacatcagtaccaagggatttgatatggtcgagctcggaagtgattccgtttaagtctccaataccgtctccgttggagtctttgaaggaagcaggccaaacttggtaaacaacggcggatttccaccaaggttcttgagactcgatagtcataaggaattaatattgtcaagagggataaaaaaattgtggaggcggagggtctttatatggcttgagcttactaaagacattctcatcactttagcgagcgcgacgctttggtatcctcctaacttcccgaaaataaaaagaggatttccgtttgcgtcatgggctccacctcctatgcacgtccataatagttatttgattgttatctgtaccccagacaagatcctgtttcagcgagtattaaatcacggcctaatttatttgagcgagaattaatttgcacatttaattctcgctgaaataactgccaacaattgagtctggggtacatgaatagaagtgacgtaattgacccccacgcttgctagccactgcggagtcacttgaaccacatgagagcccgtagggaagccagagcaatcaacgtcaaagggctcttaggaaacttgtataacccaagtgttcattacgacgctgcagagctctcacattatatgtagagtggtacggcagagtctgtagaacatacaaagctatcatgtcgcgtaatctgcacagtgcctgctttctattgcgatactcccgaagatatccctatccgcatacacggaactgtagttaatttgccgtggagtatcaatgtttgtccaacgcgccgaaactctttttgcggggctcttcttcatctaactcgtcctaagctggctcggggtatcgcataaacgtctgcgtgcattgccaagttgttttggtctgtgttgaaccttctttccaagccccatcccgaaccccggaaaattccccgattattatttccccgcaaatgcggcgtgccagaatctctcagccttgcgtcataatttagatttcgcaattatttcgtggagcaataggccgcgagcgtcctattgtgctgttggctcgaatctcatgcacaaaaatgaggatttgccagagtgtgttttccaaggcgtggccaaaacagagggtcaccagacaacagagctcaccataatacacagctttatggtcttacactaagtcctcctcctcaacaacgcggggctatacgtgtttgcagaattcttccatgcgtgatttgcaatggtgcttgcatactgcaacaatgcgtatggggtaattaaaacgagcactcgcgagcattcatcaaaggtaaaaagcattattctaagctctttctggaagcaaacttgggccgcgatcacgcaccaaaattcagccggattaagaatgggcaagcatctccccacgtcatatttgtgtggcattaggactatatatagttgatatattctcgacacactagtatccatcagcatgcctgagttcgtcgaaaacatcgagaagcctgaagaagtggaagtcattccagacatcacaaagaaaattaacactctttctgacagtgatgatggaagtggtgctttcaacgactacattgccagattcgtggagatttcgacgaatgcccagaataatgagcatcaagaaaaacacatgtcgctgaaggaaggtctgaaaactttcccgaaagctgcctgctggtcgattgtgctttccacagccatcatcatggaaggatacgataccatgctcttgaatagtttgtactcgatgcaatctttcgccaaaaaatacggccagtactacccggaaattgacgagtaccaggtccctgccaagtggcagacgtcgctttcgatgtcgacttatgtcggtgaaattgtcggactgtacattgcaggtcttgttgccgagaaatggggatacagacgtacgttgatatgcttcatggcagccgttgtgggtttaatcttcattcttttctttgcggtcgacgtgcagatgctgcttgccggcgagctgctctgtggtattgtctggggtgcattccagactcttacagtgtcatacgcctctgaagtctgtcccgttgtcctgagaatctaccttaccacttatgtcaatgcgtgctgggttatcggacagttaattgctgcttgtttgctgagaggcactatgaccctgaccaacgagtggtcatacaaaattcctttcgccgttcagtggatctggcctgtgccaatcatgattggaatctacctggccccggagtcgccctggtggctggttaaaaagaaccgggacgcagaagccaagagaagtatcatgagacttttgagtccaaacaccgaggtgccagacgttgctccgctagccgaggcaatgctgaacaaaatgcaactaaccatcaaggaagagtctgcgcgcacatccaatgtctcgtaccttgactgcttcaaacacggcaacttcagaagaacaaggattgcctccatgatctggctcatccagaatatcactggatctgtcttgatgggctattcgacctacttttacatccaggcaggacttgatagcggtatgtctttcactttctctatcatccaatatgcactgggtcttcttggaacaattgcctcgtggctgctgtcgcaaaaaatgggccgtttcgacatctatttcttgggtcttagcataaacacatgcatcctgataattgtcggaggtttgggcttctcttcatcgagcagtgcgtcttgggcaattggttcgttgctccttgtcttcacttttgtctacgactcatccatcggtcctatcacctactgtacggttgcagaaatcccttcgtcaacggtgcgtgccaagacagttgcccttgcaagaaactggtacaatctgtctcaaatcccgctctccattgtcactccatacatgctgaatccgactgcttggaactggaaagcaaaagcagcccttttatgggcaggcttgtctgtttgctcactgatctacatctggttcgagttcccagaaacaaaaggaagaacttatgctgaactggacatcctattcaaaaacggtaccagtgcccgtaagtttaagtccactcaggtggaaacattcaatcctgaggaaatgttaaaaaaaatgaataatgaggatattatacaggttgttgatggcgacctggatgccggtgcggccactgctaaagtttaaaagttgaaatgtatagtttcagttctgcgatgttattattttacaattgaatatgctatctcgaatgattttttgtaatagccgtagatgtctttattattgcatttaagggttcaaaattattcatgtgagcgttttattttacgggacaataagattttgggcgagacttccccacaaattgaataatgtgcaatatttttaagaactaaatttactggagacaatttctatgcctctcagtacacgtgcaaaaccacaaacgcctggccaaaagtcccattctaaaaggccgtatatcaggccttgtgacgcgtgcgcgttcaaaagagtacgttgcgatgctgagttcaagttggatcgcagatgcaccaactgtttagttcatggaattgagtgtacgaataacagagtcaagcagagatctggacccagaaaaatacataaaaaaactgaagaggctattaaaagcctggtagaaggaagtggcgcgttcagcaatcttcgattagccactccggcccacaacatatgtggtattatcaatcctgccacctgcgagaagagctcaatatcgcttagtgagattcgaccgtacctcagaatttataaatcccgatactatgcactctggccagtcctgtctgttgatgactgtctgaatattcttaatgaaagctcagttcacataaacgatagcgcatttttcatgctcaatgccaacaatgcgtcgttgtacgctctttcatgcgccttgtgcgctgtaattgcaagacacaccaccttctggtccctggaagaatttgatacaatggctcatttaggactcaaaagctgccctccgccagaaacttatgtaagggaggcagagcgagctatctacatgtttgaccttgacgggatactcacagaagaccaggttttaactaacttctttctgcatatatatttttcatcccttgacgaggacaacctgcaggaattggtctatctcagacaggcaatcagctgcgcacaaatgcttcaccttcgagataacgaagttttgggagaatcaccaaaagatacatcacacagatttaaaaaaatttactacttacttctcatcacagagaggtatgtctcgttcaaaaaacaactaccagtgatattagagccaacaatgccacttccaagtttggaagatgatgaagccccggatctgctacctggatttatcgaacttgcgcaagtattcagtgttcctgattgcgctttctttaacaagtactcgctccgtggcacgacagctgcacttgatcctttcagcgagagctttgacctaatgttcaaaaacgaatggatccaagacgtgcaaaccaagctctctaggtcggtcgttcaggtgcgcaaccagtctcaaaaggtcaatatattaatttctcgaacatggttgcaatcaattgcttggttaatggccaaagatcgacttcttctatcgcctacgagtgatcaagccaatttctttgatccaaggtatcctattcatatcgcgaaagactttctgatacagactaaagacatgccgtacgaggcatttgaaacaaacggtagtggtgtcatcgtgaaactaagcgaagtcgccaacgccttgcaaaccttcatccgattggctccaggatcgcccgaaacagctttagcattagacgagcttgcatatatccacggtattatcatgcggcagaactccaacagctcgctcgggcctcttatacaccctatcactgaaactttggaatctcgcaggttcggattttatcgaaatggctgcgacatttcttatccaaaaatagagtctgcagattatgaagagaacgaaaatgaccatacacccgaaatcattgcctccatgccggagaacttgaatttgacacctcttttgagcgagttcgaatttccgaggtgaacaagtattatctgctcacgtgtttggtgcattttgcaactagcgacaaatttcactggcatattagtatatcatcaaatcattaggattgctattttgtgtccactaaggcgctccaggtgcgtggaatggtgcagtattaggccctagctgtccgagaaaaaaggaagctacaccctttgatgtagtgggctctttgtgtgttgtgagaggattaaattggccagatgacactctttccaaagatgcaaataacgaggaagaaacatccagccggatgccagatgtctgtctgtgttgcaaattcgctcttgatccggaaaaagagttattgaaaagggcactgtcctgtctcgtattgcagcgtacgagtgaacctgagcgtgtcaatgcaggttcataagaagaaaattcagaagacagattgtgacatatttctcgcaagccattcactttccaagattctaggagcttagagatcttggtgaacagggctcggggcaagtccattccagctttgtacttcaacaccatgtcgaatacggcacccatgcgttcgtaggctagctcggtggttgctgggtcaacatagcttttgatcagaatgtaaagagagtctgctatctccagtaatttcattgatatgtcgggtccgttgcatctaaaggcatgatcagggaggttttgtagactgctgagaagctcatttgcaacttttagaggaaagttaatcttgaaacattcgtgttcgaccgcaatgctctcatcagaactaattagaaaattcgcagacgcaattaaccagcccaaagactggagccaggcttttgagacaataacattcactttttgagcacttgttgcaattttatccacatcaattctaatttggagcttctgttggagctcaaggagccacagcctcttcaactctgtggatccgttcaaggtaaaatcctgaaacaagttggtctctggaatctggaacatgttgtgagtgcgccctgtctcgagcactttagtatgaagctcaacatagaattgcttatccgttgcactgaaaattgcacacagagtggtgaacccgtaaagtgaatttgaggatttttcgaaatctgcttcagggagtggaattgaaggttcaagaagaacgggcaaattagttttaaagctgacaaacctttcggttaccacgagcaaatagtagatttttttctgcctatgagcctcaacgggtgacaaatgaagcagtgactgtggttcgtgcatttttaagatatctgcaagagaaacagcttcacggaggtaaataagacctttgtgctgttggccaggcgcaatagcatagtatttatgtaggaagaaggatgtcagaaggagatcaatatttggtgtgcttttcagtctatactcataaattatcctccttgcctcttgagcatataagccagcatttttgagatcgctcctggctggagtgtatgtgacgaaactgtgatattgtgcgatcgtagcgcacaatgcgcaacaaagagcatattcttgggcattgtcctcggcgatggtgatacgaaaatcatctaaaacattcgctagttgaaatacctgctccacattctttaaggggcttgaacgtgtcaaaattgaaactgtactttgaattgatactaccggccagatatcgtaaaaattcgaatgatatatctcaaggtaagggataatttgtttcaaagaaaatgcgcacacatgagcaggtagtgtagactgagcatagatttcgtcgttgagatcctctgtgtttttgactgttctagtacttttctttggaccggatttatgcctcacgcggaggttggtacatggaactttatgagtgagacagttcgagcaaataccaccggtgcatgaatctatatcgcacttcacacggcggatggcgcatgcgtcacagggtctcggccgttgtcgttttttggtggacggctgcgacatctcggtatttagataagccagcttgtacttgtatccaaaatgtggggctaaaggggagtgtatgatttttaatgatatcggtgaatgtccagaaatttggtaaatcgcacaccaaacttcaacgatggactagcgtgctaaactgatattctaccaaacaaccgtttgagtaattctgaccatgacagaggtcttgtgtatatattttaagccccgtcgatcgaccgcatcgcgactccataaaaaaattcatttcacgcgttattattatcgtatgaatatggatcccctaaacgtgaaagtgcaacagaaacttaaggagctcgaaagccttcagcagataagagacttgacgaaacacctgaatgtttcactggaagaatttgctggccaaatagaacttctgggtgaggaagcaggatgcatcgaaaccgtgacgcaaaactggatgagaatcatcagagcagtgagcctagcctctaactctttgtcgaactacaaggaagaggactatgaaacggatagaccaatgacggagcgccttgtgagatgcaagatagacgagagtcaaaaaatcatcaccaaaaattgacttacttcaatctatctaaccattgttctaaattgatcttgttgttcagttccgacaacagatcgttctccttcagcaaatcgtcacgaaggcctttatcagcctcgagataattaatatcgtccggatctaacattaagtcctgaactttagagggaagttctattttgagcgtcgataacccggtcatcattttgaagtattcattagtatgcacgtcagaaccattcaaaagcccatcccaatcagttgtcaaaaacagccttttccatatcaaggtttccaagatttcgtgatagtgcgaataaccactcaggttacttatcactcttgacggattggagtgtttgatgacagtcaacaatttgaacaagttggcgaaattgttgtgactgaattcatcaaacgtcactaatgtcagtagctggaccatctcaaacaatcccagctgttgctgcttatccatctttgccacaatatcgccgatctgactttcaaaatgtgtaaacttgttatcaaccaagtatcttgagtcttgtatgaatccatctttagtttttgaatcaattaatctcgcataattcaattgaagctcaatcatttgcaagtgtatttctatctcatccaacagattgtcgtgatcctcattcaaagctatcagtgaaagtttggcaatattcagttcgaactttttcttttcaatttcctccgactctgcagtggaaacatatgcaatcagcttttctgctgccttgaaatactcatcgttttccacgtcaagtatccaggcaaatttatagttgttgatgcttgaattcaaaaacatgcgcacgtaatctggatattgagggatgtaattgaggattagccgaatctttctggttctagcatagaatccaaaaagcacttctgcaaaacggtatccataccgctggaaacattcc

SEQ ID NO: 2 - DNA, 5978 bp, synthetic construct
  misc_feature 1..5978: note = Description of Artificial Sequence: recombinant DNA
  source 1..5978: mol_type = other DNA
caaatcaactgagaaaaatggtattcccggaaagaactcgggatacggccggattattagctaaacgagccatgttagcaaaacggttggcgaggtttatttttttatttacatttcactgcaagtcgatcgaccgtgtcgcgactaatgaataatgaatatattatgaaatgtcaagattcaactagtctatcccaatctggctccgcaaacgcttcgactgtctcataaaacaaagaaaatattcaactatatacctttattaggctgtaaaatctaattgacctcgatcagtctaccctcatatggttgcagcttgcctgtgatgttgtttgcaatattagacaggagcagctttccttgagggattgggtactcgacttctctatcagtaaagttcaaaacaacagccatcttcttttggcctttgttgctggtcttggtgtagtagaaaacctcctggttatcgtagtcttgcacctcgaaacggccaaaagtcaacgtctcaggataaagctttctggtcttgatggcatttctgtaaaagttgagcacagagttagggtcatctttctgcgaagcaacgttgatgtctttgtagttgtcgttgattctcatccaaggcttaccaccgaaaccaccgttttcagaagagtcccactgaactggagtccttgcattgtctcttgccagcaaattgatgatcttcagcagctcggccttctcctcatcagagtgctcggtctcgttgaacgcgttccagtagttaatggtgttgatatccaaatactctgaaatatcccatttcggtgaaacatttgtcataccaatctcttgaccttggtacacaaacagagtgccggtcaaggttgtctgcaacagtgccagcatctttgcagatctgctccaaaacagtttgttggaggtgtttccgaaccttgtgacacagcgtggttggtcgtggttctcgataaaaacggtggaccatgcgtcattcagctcaccggtctcgtcatcaaagatgaattctgactggttgatgatggcatccttgaagtcggtcaagttgaagcctttgtatctaaaacggtcgctcttgtcggatccaacgtcaacagtgtcgaacaggaacatcatgttcatctccttttcttttgcagagacgtacttgagagcatctgccttggagcagtgaccaacttctccgacagtcatggcatcatacctggaagtcactttctcgtacatctctttgtggaactcgtgaatacgtggaccggagttgatgagtggaccagccggctggtatttctcacctgggaaggtgattggcgcatcctcaaaagtctggacttttgagtagaggccagcagtgtcgattctgaaaccagaaacgcccttctcgtaccaaaactcaagagcagatttgtaaatggcttcacgggtctctgggttctcccagttcaaatcaggctgagtctcagcaaacaggcgaaggtagtattgcttagtcttttcgtcataagcccatgcagaaccagagaaaaaggaaagccagttgttggcattgtccttccaaatgtaccaatctctctttggattgtctctgctagatctagactccttgaaccaagcgtgctcagacgaggtgtggttaataacgaggtccagaatcaatctcatgtctctcttcttgatctcggcaagcagtctgtccatgtcctccatagtgccaaattgagggttgatggcattgtagtcagaaatatcgtatcccatatcgtccaatggcgaagcatagtgcggagaaagccagatcacatcagtaccaagggatttgatatggtcgagctcggaagtgattccgtttaagtctccaataccgtctccgttggagtctttgaaggaagcaggccaaacttggtaaacaacggcggatttccaccaaggttcttgagactcgatagtcataaggaattaatattgtcaagagggataaaaaaattgtggaggcggagggtctttatatggcttgagcttactaaagacattctcatcactttagcgagcgcgacgctttggtatcctcctaacttcccgaaaataaaaagaggatttccgtttgcgtcatgggctccacctcctatgcacgtccataatagttatttgattgttatctgtaccccagacaagatcctgtttcagcgagtattaaatcacggcctaatttatttgagcgagaattaatttgcacatttaattctcgctgaaataactgccaacaattgagtctggggtacatgaatagaagtgacgtaattgacccccacgcttgctagccactgcggagtcacttgaaccacatgagagcccgtagggaagccagagcaatcaacgtcaaagggctcttaggaaacttgtataacccaagtgttcattacgacgctgcagagctctcacattatatgtagagtggtacggcagagtctgtagaacatacaaagctatcatgtcgcgtaatctgcacagtgcctgctttctattgcgatactcccgaagatatccctatccgcatacacggaactgtagttaatttgccgtggagtatcaatgtttgtccaacgcgccgaaactctttttgcggggctcttcttcatctaactcgtcctaagctggctcggggtatcgcataaacgtctgcgtgcattgccaagttgttttggtctgtgttgaaccttctttccaagccccatcccgaaccccggaaaattccccgattattatttccccgcaaatgcggcgtgccagaatctctcagccttgcgtcataatttagatttcgcaattatttcgtggagcaataggccgcgagcgtcctattgtgctgttggctcgaatctcatgcacaaaaatgaggatttgccagagtgtgttttccaaggcgtggccaaaacagagggtcaccagacaacagagctcaccataatacacagctttatggtcttacactaagtcctcctcctcaacaacgcggggctatacgtgtttgcagaattcttccatgcgtgatttgcaatggtgcttgcatactgcaacaatgcgtatggggtaattaaaacgagcactcgcgagcattcatcaaaggtaaaaagcattattctaagctctttctggaagcaaacttgggccgcgatcacgcaccaaaattcagccggattaagaatgggcaagcatctccccacgtcatatttgtgtggcattaggactatatatagttgatatattctcgacacactagtatccatcagcatgcctgagttcgtcgaaaacatcgagaagcctgaagaagtggaagtcattccagacatcacaaagaaaattaacactctttctgacagtgatgatggaagtggtgctttcaacgactacattgccagattcgtggagatttcgacgaatgcccagaataatgagcatcaagaaaaacacatgtcgctgaaggaaggtctgaaaactttcccgaaagctgcctgctggtcgattgtgctttccacagccatcatcatggaaggatacgataccatgctcttgaatagtttgtactcgatgcaatctttcgccaaaaaatacggccagtactacccggaaattgacgagtaccaggtccctgccaagtggcagacgtcgctttcgatgtcgacttatgtcggtgaaattgtcggactgtacattgcaggtcttgttgccgagaaatggggatacagacgtacgttgatatgcttcatggcagccgttgtgggtttaatcttcattcttttctttgcggtcgacgtgcagatgctgcttgccggcgagctgctctgtggtattgtctggggtgcattccagactcttacagtgtcatacgcctctgaagtctgtcccgttgtcctgagaatctaccttaccacttatgtcaatgcgtgctgggttatcggacagttaattgctgcttgtttgctgagaggcactatgaccctgaccaacgagtggtcatacaaaattcctttcgccgttcagtggatctggcctgtgccaatcatgattggaatctacctggccccggagtcgccctggtggctggttaaaaagaaccgggacgcagaagccaagagaagtatcatgagacttttgagtccaaacaccgaggtgccagacgttgctccgctagccgaggcaatgctgaacaaaatgcaactaaccatcaaggaagagtctgcgcgcacatccaatgtctcgtaccttgactgcttcaaacacggcaacttcagaagaacaaggattgcctccatgatctggctcatccagaatatcactggatctgtcttgatgggctattcgacctacttttacatccaggcaggacttgatagcggtatgtctttcactttctctatcatccaatatgcactgggtcttcttggaacaattgcctcgtggctgctgtcgcaaaaaatgggccgtttcgacatctatttcttgggtcttagcataaacacatgcatcctgataattgtcggaggtttgggcttctcttcatcgagcagtgcgtcttgggcaattggttcgttgctccttgtcttcacttttgtctacgactcatccatcggtcctatcacctactgtacggttgcagaaatcccttcgtcaacggtgcgtgccaagacagttgcccttgcaagaaactggtacaatctgtctcaaatcccgctctccattgtcactccatacatgctgaatccgactgcttggaactggaaagcaaaagcagcccttttatgggcaggcttgtctgtttgctcactgatctacatctggttcgagttcccagaaacaaaaggaagaacttatgctgaactggacatcctattcaaaaacggtaccagtgcccgtaagtttaagtccactcaggtggaaacattcaatcctgaggaaatgttaaaaaaaatgaataatgaggatattatacaggttgttgatggcgacctggatgccggtgcggccactgctaaagtttaaaagttgaaatgtatagtttcagttctgcgatgttattattttacaattgaatatgctatctcgaatgattttttgtaatagccgtagatgtctttattattgcatttaagggttcaaaattattcatgtgagcgttttattttacgggacaataagattttgggcgagacttccccacaaattgaataatgtgcaatatttttaagaactaaatttactggagacaatttctatgcctctcagtacacgtgcaaaaccacaaacgcctggccaaaagtcccattctaaaaggccgtatatcaggccttgtgacgcgtgcgcgttcaaaagagtacgttgcgatgctgagttcaagttggatcgcagatgcaccaactgtttagttcatggaattgagtgtacgaataacagagtcaagcagagatctggacccagaaaaatacataaaaaaactgaagaggctattaaaagcctggtagaaggaagtggcgcgttcagcaatcttcgattagccactccggcccacaacatatgtggtattatcaatcctgccacctgcgagaagagctcaatatcgcttagtgagattcgaccgtacctcagaatttataaatcccgatactatgcactctggccagtcctgtctgttgatgactgtctgaatattcttaatgaaagctcagttcacataaacgatagcgcatttttcatgctcaatgccaacaatgcgtcgttgtacgctctttcatgcgccttgtgcgctgtaattgcaagacacaccaccttctggtccctggaagaatttgatacaatggctcatttaggactcaaaagctgccctccgccagaaacttatgtaagggaggcagagcgagctatctacatgtttgaccttgacgggatactcacagaagaccagg

SEQ ID NO: 3 - DNA, 1021 bp, synthetic construct
  misc_feature 1..1021: note = Description of Artificial Sequence: recombinant DNA
  source 1..1021: mol_type = other DNA
ttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggtattcccggaa

SEQ ID NO: 4 - DNA, 5733 bp, synthetic construct
  misc_feature 1..5733: note = Description of Artificial Sequence: recombinant DNA
  source 1..5733: mol_type = other DNA
tgggcaggcttgtctgtttgctcactgatctacatctggttcgagttcccagaaacaaaaggaagaacttatgctgaactggacatcctattcaaaaacggtaccagtgcccgtaagtttaagtccactcaggtggaaacattcaatcctgaggaaatgttaaaaaaaatgaataatgaggatattatacaggttgttgatggcgacctggatgccggtgcggccactgctaaagtttaaaagttgaaatgtatagtttcagttctgcgatgttattattttacaattgaatatgctatctcgaatgattttttgtaatagccgtagatgtctttattattgcatttaagggttcaaaattattcatgtgagcgttttattttacgggacaataagattttgggcgagacttccccacaaattgaataatgtgcaatatttttaagaactaaatttactggagacaatttctatgcctctcagtacacgtgcaaaaccacaaacgcctggccaaaagtcccattctaaaaggccgtatatcaggccttgtgacgcgtgcgcgttcaaaagagtacgttgcgatgctgagttcaagttggatcgcagatgcaccaactgtttagttcatggaattgagtgtacgaataacagagtcaagcagagatctggacccagaaaaatacataaaaaaactgaagaggctattaaaagcctggtagaaggaagtggcgcgttcagcaatcttcgattagccactccggcccacaacatatgtggtattatcaatcctgccacctgcgagaagagctcaatatcgcttagtgagattcgaccgtacctcagaatttataaatcccgatactatgcactctggccagtcctgtctgttgatgactgtctgaatattcttaatgaaagctcagttcacataaacgatagcgcatttttcatgctcaatgccaacaatgcgtcgttgtacgctctttcatgcgccttgtgcgctgtaattgcaagacacaccaccttctggtccctggaagaatttgatacaatggctcatttaggactcaaaagctgccctccgccagaaacttatgtaagggaggcagagcgagctatctacatgtttgaccttgacgggatactcacagaagaccaggttttaactaacttctttctgcatatatatttttcatcccttgacgaggacaacctgcaggaattggtctatctcagacaggcaatcagctgcgcacaaatgcttcaccttcgagataacgaagttttgggagaatcaccaaaagatacatcacacagatttaaaaaaatttactacttacttctcatcacagagaggtatgtctcgttcaaaaaacaactaccagtgatattagagccaacaatgccacttccaagtttggaagatgatgaagccccggatctgctacctggatttatcgaacttgcgcaagtattcagtgttcctgattgcgctttctttaacaagtactcgctccgtggcacgacagctgcacttgatcctttcagcgagagctttgacctaatgttcaaaaacgaatggatccaagacgtgcaaaccaagctctctaggtcggtcgttcaggtgcgcaaccagtctcaaaaggtcaatatattaatttctcgaacatggttgcaatcaattgcttggttaatggccaaagatcgacttcttctatcgcctacgagtgatcaagccaatttctttgatccaaggtatcctattcatatcgcgaaagactttctgatacagactaaagacatgccgtacgaggcatttgaaacaaacggtagtggtgtcatcgtgaaactaagcgaagtcgccaacgccttgcaaaccttcatccgattggctccaggatcgcccgaaacagctttagcattagacgagcttgcatatatccacggtattatcatgcggcagaactccaacagctcgctcgggcctcttatacaccctatcactgaaactttggaatctcgcaggttcggattttatcgaaatggctgcgacatttcttatccaaaaatagagtctgcagattatgaagagaacgaaaatgaccatacacccgaaatcattgcctccatgccggagaacttgaatttgacacctcttttgagcgagttcgaatttccgaggtgaacaagtattatctgctcacgtgtttggtgcattttgcaactagcgacaaatttcactggcatattagtatatcatcaaatcattaggattgctattttgtgtccactaaggcgctccaggtgcgtggaatggtgcagtattaggccctagctgtccgagaaaaaaggaagctacaccctttgatgtagtgggctctttgtgtgttgtgagaggattaaattggccagatgacactctttccaaagatgcaaataacgaggaagaaacatccagccggatgccagatgtctgtctgtgttgcaaattcgctcttgatccggaaaaagagttattgaaaagggcactgtcctgtctcgtattgcagcgtacgagtgaacctgagcgtgtcaatgcaggttcataagaagaaaattcagaagacagattgtgacatatttctcgcaagccattcactttccaagattctaggagcttagagatcttggtgaacagggctcggggcaagtccattccagctttgtacttcaacaccatgtcgaatacggcacccatgcgttcgtaggctagctcggtggttgctgggtcaacatagcttttgatcagaatgtaaagagagtctgctatctccagtaatttcattgatatgtcgggtccgttgcatctaaaggcatgatcagggaggttttgtagactgctgagaagctcatttgcaacttttagaggaaagttaatcttgaaacattcgtgttcgaccgcaatgctctcatcagaactaattagaaaattcgcagacgcaattaaccagcccaaagactggagccaggcttttgagacaataacattcactttttgagcacttgttgcaattttatccacatcaattctaatttggagcttctgttggagctcaaggagccacagcctcttcaactctgtggatccgttcaaggtaaaatcctgaaacaagttggtctctggaatctggaacatgttgtgagtgcgccctgtctcgagcactttagtatgaagctcaacatagaattgcttatccgttgcactgaaaattgcacacagagtggtgaacccgtaaagtgaatttgaggatttttcgaaatctgcttcagggagtggaattgaaggttcaagaagaacgggcaaattagttttaaagctgacaaacctttcggttaccacgagcaaatagtagatttttttctgcctatgagcctcaacgggtgacaaatgaagcagtgactgtggttcgtgcatttttaagatatctgcaagagaaacagcttcacggaggtaaataagacctttgtgctgttggccaggcgcaatagcatagtatttatgtaggaagaaggatgtcagaaggagatcaatatttggtgtgcttttcagtctatactcataaattatcctccttgcctcttgagcatataagccagcatttttgagatcgctcctggctggagtgtatgtgacgaaactgtgatattgtgcgatcgtagcgcacaatgcgcaacaaagagcatattcttgggcattgtcctcggcgatggtgatacgaaaatcatctaaaacattcgctagttgaaatacctgctccacattctttaaggggcttgaacgtgtcaaaattgaaactgtactttgaattgatactaccggccagatatcgtaaaaattcgaatgatatatctcaaggtaagggataatttgtttcaaagaaaatgcgcacacatgagcaggtagtgtagactgagcatagatttcgtcgttgagatcctctgtgtttttgactgttctagtacttttctttggaccggatttatgcctcacgcggaggttggtacatggaactttatgagtgagacagttcgagcaaataccaccggtgcatgaatctatatcgcacttcacacggcggatggcgcatgcgtcacagggtctcggccgttgtcgttttttggtggacggctgcgacatctcggtatttagataagccagcttgtacttgtatccaaaatgtggggctaaaggggagtgtatgatttttaatgatatcggtgaatgtccagaaatttggtaaatcgcacaccaaacttcaacgatggactagcgtgctaaactgatattctaccaaacaaccgtttgagtaattctgaccatgacagaggtcttgtgtatatattttaagccccgtcgatcgaccgcatcgcgactccataaaaaaattcatttcacgcgttattattatcgtatgaatatggatcccctaaacgtgaaagtgcaacagaaacttaaggagctcgaaagccttcagcagataagagacttgacgaaacacctgaatgtttcactggaagaatttgctggccaaatagaacttctgggtgaggaagcaggatgcatcgaaaccgtgacgcaaaactggatgagaatcatcagagcagtgagcctagcctctaactctttgtcgaactacaaggaagaggactatgaaacggatagaccaatgacggagcgccttgtgagatgcaagatagacgagagtcaaaaaatcatcaccaaaaattgacttacttcaatctatctaaccattgttctaaattgatcttgttgttcagttccgacaacagatcgttctccttcagcaaatcgtcacgaaggcctttatcagcctcgagataattaatatcgtccggatctaacattaagtcctgaactttagagggaagttctattttgagcgtcgataacccggtcatcattttgaagtattcattagtatgcacgtcagaaccattcaaaagcccatcccaatcagttgtcaaaaacagccttttccatatcaaggtttccaagatttcgtgatagtgcgaataaccactcaggttacttatcactcttgacggattggagtgtttgatgacagtcaacaatttgaacaagttggcgaaattgttgtgactgaattcatcaaacgtcactaatgtcagtagctggaccatctcaaacaatcccagctgttgctgcttatccatctttgccacaatatcgccgatctgactttcaaaatgtgtaaacttgttatcaaccaagtatcttgagtcttgtatgaatccatctttagtttttgaatcaattaatctcgcataattcaattgaagctcaatcatttgcaagtgtatttctatctcatccaacagattgtcgtgatcctcattcaaagctatcagtgaaagtttggcaatattcagttcgaactttttcttttcaatttcctccgactctgcagtggaaacatatgcaatcagcttttctgctgccttgaaatactcatcgttttccacgtcaagtatccaggcaaatttatagttgttgatgcttgaattcaaaaacatgcgcacgtaatctggatattgagggatgtaattgaggattagccgaatctttctggttctagcatagaatccaaaaagcacttctgcaaaacggtatccataccgctggaaacattcctagtttatgttgtatctatg

SEQ ID NO: 5 - DNA, 1000 bp, synthetic construct
  misc_feature 1..1000: note = Description of Artificial Sequence: recombinant DNA
  source 1..1000: mol_type = other DNA
cataccgctggaaacattcctagtttatgttgtatctatgaatatttttttacatggtggatttgttccaatctagttgttagttatttgtcgttatagtcactaatacagttaccatcggaatcttttcatttctttttatacgtacgtatatgtactagatgaagaatgcgacaaggccgaccaacagcaatggtgcttggtaccaaagtttggaaggtgctaccgaattggccgatgatattgaagtggagttgtcattctcctcattggttgctccagtagtggtaaaattaacggggatatcttcggtggaagtgtcaatatcaccgttccaacaataattctcaaaacatcttctacattgctctgtttgtgttgcattggatctctcctgctgtctacgaatgattgcacatccaacacaagtacgccattcttcatcaatagtcatgttcaaacgagacgacacttcaaaaccgttttggatcattcctctcttctcggattcagagtagtctaacttgaaagttgaagtgttactccaatacgagaaaggtctattggccagataaacaaccaggggaggaatgtgatccgtgccttcaacaatgtctgtcaaatttctagcatcacaaccaaagaaggttggcttactcgaaagattcaagtttaggaaagtggtggtatcaggtacataagggaacgttgttccatttgcttgagaagaaaactgtctcatgtaggtgttgactaatgatgaaccgtttggccaagagaggtcagtgtctgcactgttgtcaaacgcaaagatgatatcaacgtcacgctccttttgaagtagaggctgcagagggacgttttggttatcctctccaccatcaaccaagtaaagggtgtcattttccgcaatggcacctactcctgcatacgtacttttgtagaaagggttgggggaatagatagcaatgtcgtcttcgtcttcgctaagtccagtcag

SEQ ID NO: 6 - DNA, 20 bp, synthetic construct
  misc_feature 1..20: note = Description of Artificial Sequence: primer
  source 1..20: mol_type = other DNA
ttgtcagcttaaaggactcc

SEQ ID NO: 7 - DNA, 40 bp, synthetic construct
  misc_feature 1..40: note = Description of Artificial Sequence: primer
  source 1..40: mol_type = other DNA
ttccgggaataccatttttctcagttgatttgtttgtggg

SEQ ID NO: 8 - DNA, 40 bp, synthetic construct
  misc_feature 1..40: note = Description of Artificial Sequence: primer
  source 1..40: mol_type = other DNA
caaatcaactgagaaaaatggtattcccggaaagaactcg

SEQ ID NO: 9 - DNA, 20 bp, synthetic construct
  misc_feature 1..20: note = Description of Artificial Sequence: primer
  source 1..20: mol_type = other DNA
cctggtcttctgtgagtatc

SEQ ID NO: 10 - DNA, 20 bp, synthetic construct
  misc_feature 1..20: note = Description of Artificial Sequence: primer
  source 1..20: mol_type = other DNA
tgggcaggcttgtctgtttg

SEQ ID NO: 11 - DNA, 40 bp, synthetic construct
  misc_feature 1..40: note = Description of Artificial Sequence: primer
  source 1..40: mol_type = other DNA
catagatacaacataaactaggaatgtttccagcggtatg

SEQ ID NO: 12 - DNA, 40 bp, synthetic construct
  misc_feature 1..40: note = Description of Artificial Sequence: primer
  source 1..40: mol_type = other DNA
cataccgctggaaacattcctagtttatgttgtatctatg

SEQ ID NO: 13 - DNA, 19 bp, synthetic construct
  misc_feature 1..19: note = Description of Artificial Sequence: primer
  source 1..19: mol_type = other DNA
ctgactggacttagcgaag

SEQ ID NO: 14 - protein, 571 aa, synthetic construct
  REGION 1..571: note = Description of Artificial Sequence: recombinant peptide
GAPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPG

SEQ ID NO: 15 - DNA, 5521 bp, synthetic construct
  misc_feature 1..5521: note = Description of Artificial Sequence: plasmid
  source 1..5521: mol_type = other DNA
agatctgcttgcccattcttaatccggctgaattttggtgcgtgatcgcggcccaagtttgcttccagaaagagcttagaataatgctttttacctttgatgaatgctcgcgagtgctcgttttaattaccccatacgcattgttgcagtatgcaagcaccattgcaaatcacgcatggaagaattctgcaaacacgtatagccccgcgttgttgaggaggaggacttagtgtaagaccataaagctgtgtattatggtgagctctgttgtctggtgaccctctgttttggccacgccttggaaaacacactctggcaaatcctcatttttgtgcatgagattcgagccaacagcacaataggacgctcgcggcctattgctccacgaaataattgcgaaatctaaattatgacgcaaggctgagagattctggcacgccgcatttgcggggaaataataatcggggaattttccggggttcgggatggggcttggaaagaaggttcaacacagaccaaaacaacttggcaatgcacgcagacgtttatgcgataccccgagccagcttaggacgagttagatgaagaagagccccgcaaaaagagtttcggcgcgttggacaaacattgatactccacggcaaattaactacagttccgtgtatgcggatagggatatcttcgggagtatcgcaatagaaagcaggcactgtgcagattacgcgacatgatagctttgtatgttctacagactctgccgtaccactctacatataatgtgagagctctgcagcgtcgtaatgaacacttgggttatacaagtttcctaagagccctttgacgttgattgctctggcttccctacgggctctcatgtggttcaagtgactccgcagtggctagcaagcgtgggggtcaattacgtcacttctattcatgtaccccagactcaattgttggcagttatttcagcgagaattaaatgtgcaaattaattctcgctcaaataaattaggccgtgatttaatactcgctgaaacaggatcttgtctggggtacagataacaatcaaataactattatggacgtgcataggaggtggagcccatgacgcaaacggaaatcctctttttattttcgggaagttaggaggataccaaagcgtcgcgctcgctaaagtgatgagaatgtctttagtaagctcaagccatataaagaccctccgcctccacaatttttttatccctcttgacaatattaattccttatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctggagcaccaggtgccccaggcttgcaaggagctcctggcctgcagggtatgccaggagagagaggtgccgctggcttgccaggtccaaagggagagagaggtgatgccggtccaaagggcgctgatggagcaccgggtgctccaggtttgcagggaatgcctggcgaaagaggcgccgccggattgccaggtccaaaaggcgaacgtggagatgctggtcctaagggtgcagatggagctccaggtaaagacggcgttagaggtctggcaggtccaattggacctccaggtgagagaggagctgcaggattgccaggcccaaagggtgaacgtggcgacgccggtccaaagggagcagatggagctccgggtaaagatggagtgagaggactggcaggccctatcggtccaccaggaccagctggtgccccaggcgctcctggtttgcaaggtatgcctggagagagaggtgcagctggactgccaggcccaaaaggtgaaagaggtgatgccggaccaaaaggcgccgatggtgctccaggtaaagatggcgttagaggtcttgctggtccacccggagcccctggcttgcagggcgcaccaggattgcagggtatgccaggcgagagaggagctgcaggtctgcctggtccaaaaggtgaaagaggagatgctggtcctaagggtgctgacggcgcccctggagctccaggattgcagggaatgccaggtgagagaggcgctgccggtttgccaggacctaagggtgaaagaggtgatgctggcccaaaaggtgccgatggagctccaggaaaagatggtgttagaggactggccggtcctatcggccctcctggagaaagaggagctgctggtttgcctggcccaaaaggcgagagaggtgacgctggcccaaaaggagccgacggtgctcctggcaaggacggagtgagaggcctggccggtcctattggtccaccaggacctgctggcgctccaggagctcctggtttgcagggtatgccaggtgaaagaggagccgcaggcctgccaggtcctaagggtgagagaggtgacgccggtcctaaaggcgctgatggagctcctggtaaagatggcgttagaggtttggccggacccccaggtgccccaggtctgcaaggagctccaggtcttcagggtatgccaggcgaaagaggagctgctggtttgccaggcccaaagggtgaaagaggagatgccggcccaaaaggagctgacggtgcaccaggcgctccaggcctgcagggtatgccaggagaaagaggtgctgctggtttgccaggaccaaagggagaaagaggcgacgccggtccaaagggtgcagatggagctcctggcaaagacggagtgagaggcttggcaggtccaatcggaccgccaggcgagagaggtgctgccggcttgccaggtccaaagggtgaaagaggagacgcaggtcctaaaggtgctgacggcgcaccaggaaaagatggtgttagaggtctggctggacctatcggcccaccaggaccagctggtgctccaggtgctcctggccttcagggcatgccaggagaaagaggtgctgccggtctgcctggacctaagggcgaaagaggcgatgcaggtccaaagggtgctgatggagctcctggtaaagatggtgttagaggattggcaggcccacccgggtaatagtctagagtcgactgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcactagtcccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgcgacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtcgccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 16 - DNA, 1261 bp, synthetic construct
  misc_feature 1..1261: note = Description of Artificial Sequence: recombinant DNA
  source 1..1261: mol_type = other DNA
gcttgcccattcttaatccggctgaattttggtgcgtgatcgcggcccaagtttgcttccagaaagagcttagaataatgctttttacctttgatgaatgctcgcgagtgctcgttttaattaccccatacgcattgttgcagtatgcaagcaccattgcaaatcacgcatggaagaattctgcaaacacgtatagccccgcgttgttgaggaggaggacttagtgtaagaccataaagctgtgtattatggtgagctctgttgtctggtgaccctctgttttggccacgccttggaaaacacactctggcaaatcctcatttttgtgcatgagattcgagccaacagcacaataggacgctcgcggcctattgctccacgaaataattgcgaaatctaaattatgacgcaaggctgagagattctggcacgccgcatttgcggggaaataataatcggggaattttccggggttcgggatggggcttggaaagaaggttcaacacagaccaaaacaacttggcaatgcacgcagacgtttatgcgataccccgagccagcttaggacgagttagatgaagaagagccccgcaaaaagagtttcggcgcgttggacaaacattgatactccacggcaaattaactacagttccgtgtatgcggatagggatatcttcgggagtatcgcaatagaaagcaggcactgtgcagattacgcgacatgatagctttgtatgttctacagactctgccgtaccactctacatataatgtgagagctctgcagcgtcgtaatgaacacttgggttatacaagtttcctaagagccctttgacgttgattgctctggcttccctacgggctctcatgtggttcaagtgactccgcagtggctagcaagcgtgggggtcaattacgtcacttctattcatgtaccccagactcaattgttggcagttatttcagcgagaattaaatgtgcaaattaattctcgctcaaataaattaggccgtgatttaatactcgctgaaacaggatcttgtctggggtacagataacaatcaaataactattatggacgtgcataggaggtggagcccatgacgcaaacggaaatcctctttttattttcgggaagttaggaggataccaaagcgtcgcgctcgctaaagtgatgagaatgtctttagtaagctcaagccatataaagaccctccgcctccacaatttttttatccctcttgacaatattaattcctt

SEQ ID NO: 17 - DNA, 267 bp, synthetic construct
  misc_feature 1..267: note = Description of Artificial Sequence: recombinant DNA
  source 1..267: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 18 - DNA, 1713 bp, synthetic construct
  misc_feature 1..1713: note = Description of Artificial Sequence: recombinant DNA
  source 1..1713: mol_type = other DNA
ggagcaccaggtgccccaggcttgcaaggagctcctggcctgcagggtatgccaggagagagaggtgccgctggcttgccaggtccaaagggagagagaggtgatgccggtccaaagggcgctgatggagcaccgggtgctccaggtttgcagggaatgcctggcgaaagaggcgccgccggattgccaggtccaaaaggcgaacgtggagatgctggtcctaagggtgcagatggagctccaggtaaagacggcgttagaggtctggcaggtccaattggacctccaggtgagagaggagctgcaggattgccaggcccaaagggtgaacgtggcgacgccggtccaaagggagcagatggagctccgggtaaagatggagtgagaggactggcaggccctatcggtccaccaggaccagctggtgccccaggcgctcctggtttgcaaggtatgcctggagagagaggtgcagctggactgccaggcccaaaaggtgaaagaggtgatgccggaccaaaaggcgccgatggtgctccaggtaaagatggcgttagaggtcttgctggtccacccggagcccctggcttgcagggcgcaccaggattgcagggtatgccaggcgagagaggagctgcaggtctgcctggtccaaaaggtgaaagaggagatgctggtcctaagggtgctgacggcgcccctggagctccaggattgcagggaatgccaggtgagagaggcgctgccggtttgccaggacctaagggtgaaagaggtgatgctggcccaaaaggtgccgatggagctccaggaaaagatggtgttagaggactggccggtcctatcggccctcctggagaaagaggagctgctggtttgcctggcccaaaaggcgagagaggtgacgctggcccaaaaggagccgacggtgctcctggcaaggacggagtgagaggcctggccggtcctattggtccaccaggacctgctggcgctccaggagctcctggtttgcagggtatgccaggtgaaagaggagccgcaggcctgccaggtcctaagggtgagagaggtgacgccggtcctaaaggcgctgatggagctcctggtaaagatggcgttagaggtttggccggacccccaggtgccccaggtctgcaaggagctccaggtcttcagggtatgccaggcgaaagaggagctgctggtttgccaggcccaaagggtgaaagaggagatgccggcccaaaaggagctgacggtgcaccaggcgctccaggcctgcagggtatgccaggagaaagaggtgctgctggtttgccaggaccaaagggagaaagaggcgacgccggtccaaagggtgcagatggagctcctggcaaagacggagtgagaggcttggcaggtccaatcggaccgccaggcgagagaggtgctgccggcttgccaggtccaaagggtgaaagaggagacgcaggtcctaaaggtgctgacggcgcaccaggaaaagatggtgttagaggtctggctggacctatcggcccaccaggaccagctggtgctccaggtgctcctggccttcagggcatgccaggagaaagaggtgctgccggtctgcctggacctaagggcgaaagaggcgatgcaggtccaaagggtgctgatggagctcctggtaaagatggtgttagaggattggcaggcccacccggg

SEQ ID NO: 19 - DNA, 5269 bp, synthetic construct
  misc_feature 1..5269: note = Description of Artificial Sequence: plasmid
  source 1..5269: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaagaatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctggagctccaggtgctccaggccttcagggtgctccaggtttgcagggtatgccaggagagagaggagctgccggtttgcctggcccaaagggtgagagaggtgacgctggtcctaaaggtgctgacggagctccaggagctcctggactgcaaggcatgcctggtgaaagaggtgcagctggcctgccaggaccaaaaggagaaagaggcgatgcaggcccaaagggagcagatggtgccccaggtaaagacggtgttagaggcttggctggacctatcggacctccaggtgaaagaggtgcagctggcctgccaggaccaaaaggagaaagaggcgatgcaggcccaaagggagcagatggtgccccaggtaaagacggtgttagaggcttggctggacctatcggaccacctggtccagctggagcccctggtgctccaggtttgcagggtatgccaggagagagaggagctgccggtttgcctggtccaaagggcgaaagaggtgatgccggaccgaaaggcgctgatggcgccccaggcaaggatggcgtgagaggtctggccggtccacccggtgctccaggccttcagggtgctccaggtttgcagggtatgccaggagagagaggagctgccggtttgcctggcccaaagggtgagagaggtgacgctggtcctaaaggtgctgacggagctccaggagctcctggactgcaaggcatgcctggtgaaagaggtgcagctggcctgccaggaccaaaaggagaaagaggcgatgcaggcccaaagggagcagatggtgccccaggtaaagacggtgttagaggcttggctggacctatcggacctccaggtgaaagaggtgcagctggcctgccaggaccaaaaggagaaagaggcgatgcaggcccaaagggagcagatggtgccccaggtaaagacggtgttagaggcttggctggacctatcggaccacctggtccagctggagcccctggtgctccaggtttgcagggtatgccaggagagagaggagctgccggtttgcctggtccaaagggcgaaagaggtgatgccggaccgaaaggcgctgatggcgccccaggcaaggatggcgtgagaggtctggccggtccacccggtgctccaggccttcagggtgctccaggtttgcagggtatgccaggagagagaggagctgccggtttgcctggcccaaagggtgagagaggtgacgctggtcctaaaggtgctgacggagctccaggagctcctggactgcaaggcatgcctggtgaaagaggtgcagctggcctgccaggaccaaaaggagaaagaggcgatgcaggcccaaagggagcagatggtgccccaggtaaagacggtgttagaggcttggctggacctatcggacctccaggtgaaagaggtgcagctggcctgccaggaccaaaaggagaaagaggcgatgcaggcccaaagggagcagatggtgccccaggtaaagacggtgttagaggcttggctggacctatcggaccacctggtccagctggagcccctggtgctccaggtttgcagggtatgccaggagagagaggagctgccggtttgcctggtccaaagggcgaaagaggtgatgccggaccgaaaggcgctgatggcgccccaggcaaggatggcgtgagaggtctggccggtccacccgggtaagcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacgcggcccgacgggtccgaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc